(19) United States
(12) Patent Application Publication
(10) Pub. No.: US 2024/0110175 A1
(43) Pub. Date: Apr. 4, 2024

(54) COMPOSITION AND METHOD FOR HIGH-MULTIPLEXED GENOME ENGINEERING USING SYNTHETIC CRISPR ARRAYS

(71) Applicant: The Board of Trustees of the Leland Stanford Junior University, Stanford, CA (US)

(72) Inventors: Jens Peter MAGNUSSON, Stanford, CA (US); Lei S. QI, Stanford, CA (US)

(21) Appl. No.: 18/261,953

(22) PCT Filed: Jan. 18, 2022
(86) PCT No.: PCT/US22/12822

§ 371 Date: Jul. 18, 2023

Related U.S. Application Data
(60) Provisional application No. 63/139,095, filed on Jan. 19, 2021.

(51) Int. Cl. C12N 15/10; C12N 9/22; C12N 15/11; C12N 15/90

(52) U.S. Cl. CPC C12N 15/1082; C12N 9/22; C12N 15/11; C12N 15/907; C12N 2310/20; C12N 2800/80

(57) ABSTRACT

The present disclosure generally relates to compositions and methods simultaneous, multi-mode gene expression regulation (e.g., simultaneous upregulation and down regulation of multiple target genes). The present disclosure further relates to novel constructs for engineered multiplex CRISPR arrays.

### CROSS-REFERENCE TO RELATED APPLICATIONS

This application claims priority to U.S. Provisional Patent Application No. 63/139,095, filed Jan. 19, 2021, the disclosure of which is incorporated by reference herein in its entirety, including any drawings.

### FIELD OF THE DISCLOSURE

The present disclosure generally relates to compositions and methods simultaneous, multi-mode gene expression regulation (e.g., simultaneous upregulation and down regulation of multiple target genes). The present disclosure further relates to novel constructs for engineered multiplex CRISPR arrays.

### BACKGROUND

Most complex cell behaviors are regulated by the coordinated action of many genes. For example, precise cell identity engineering often requires co-expression of multiple transcription factors in the same cells. The ability to efficiently up-regulate a set of genes while down-regulating another set of genes also determines the successful outcome of cell reprogramming and cell therapy. One long-term goal in biology is the ability to control cell identity and behavior with high precision and high throughput. To reach this goal, one prerequisite will be the ability to control expression of many genes at the same time, with each gene activated or silenced in parallel.

Past work has shown some capability to either up-regulate or down-regulate a few genes, typically limited to about 3-4 genes, at a time. Some examples include introduction of expression vectors carrying cDNA for each gene of interest where each cDNA is encoded on its own plasmid; gene repression using RNA interference; gene knockout using gene-editing tools such as CRISPR/Cas, TALENs or Zinc-finger nucleases; or gene activation, inhibition, or knockdown using modified versions of the CRISPR/Cas system. However, none of these methods is capable of simultaneously regulating more than a handful of genes in each cell. Further, there is no method for simultaneously activating and repressing many genes in the same cells. Some methods use complementary DNA (cDNAs) to overexpress a few genes while using CRISPR gene knockout or RNAi knockdown to silence a few genes. It is highly labor intensive and unsuitable for large-scale cell engineering-based therapy or in vivo gene therapy.

The compositions and methods described herein enable use of a compact single CRISPR array to control many genes (e.g., 30 or more genes at one time) for multiple modes of genome engineering (e.g., simultaneous up- and down-regulation) in the same cells, using a minimal amount of molecular compositions.

### BRIEF SUMMARY

Provided herein, among others, are engineered multiplex Cluster Regularly Interspaced Short Palindromic Repeat (CRISPR) arrays. In some embodiments, an engineered multiplex CRISPR) arrays provided herein comprises more than one CRISPR RNA (crRNA). In some embodiments, each of the more than one crRNAs comprises a repeat sequence and a spacer. In some embodiments, the spacer is configured to hybridize to a specific target nucleic acid of a plurality of target nucleic acids. In other embodiments, the repeat sequence in each of the more than one crRNAs is preceded by a separator sequence.

In some embodiments, at least a portion of the more than one crRNAs comprise a Cas12a repeat sequence. In some embodiments, the engineered multiplex CRISPR array is capable of upregulating the expression of the plurality of target nucleic acids simultaneously.

In other embodiments, at least a portion of the more than one crRNAs comprise a Cas13 repeat sequence. In such embodiments, the engineered multiplex CRISPR array is capable of downregulating the expression of the plurality of target nucleic acids simultaneously.

In still other embodiments, at least a portion of the more than one crRNAs comprise a Cas12a repeat sequence and at least a portion of the more than one crRNAs comprise a Cas13 repeat sequence. In those embodiments, the engineered multiplex CRISPR array is capable of upregulating and downregulating the expression of the plurality of target nucleic acids simultaneously. In some embodiments, the plurality of target nucleic acids comprises at least 4 different target nucleic acids. And in certain embodiments, the Cas13 protein comprises a Cas13d protein and a Cas13b protein.

In some embodiments, the average length of the crRNA of the engineered multiplex CRISPR arrays provided herein is about 30 to about 70 nucleotides. In certain embodiments, the average length of the crRNA is about 50 nucleotides.

In some embodiments, the separator sequence of the engineered multiplex CRISPR arrays provided herein comprises an AT-rich sequence. In some embodiments, the separator sequence is about 3 to about 8 nucleotides in length.

In some embodiments, the plurality of target nucleic acids described herein are RNAs. In other embodiments, the plurality of target nucleic acids described herein are double-stranded DNAs (dsDNAs).

Further provided herein are nucleic acids encoding the engineered multiplex CRISPR arrays described herein.

Additionally, the present disclosure also provides vectors comprising the nucleic acids. In some embodiments, the vectors provided herein further comprises a promoter. In some embodiments, the promoter comprises a polymerase II promoter. In certain embodiments, the polymerase II promoter comprises a CAG promoter, an avPGK promoter, an EF1a promoter, and a SFFV promoter.

In other embodiments, the vectors provided herein further comprises a reporter gene. In some embodiments, the reporter gene comprises BFP, GFP, and mCherry.

In some embodiments, the vectors provided herein comprises a lentiviral vector, Adeno-associated viral vector, and piggyBac vector.

Also provided herein, among other, is a method of making a collection of engineered multiplex CRISPR arrays of the present disclosure. In some embodiments, the method of making a collection of engineered multiplex CRISPR arrays comprises providing more than one crRNAs, wherein each of the more than one crRNAs comprises a 5′ oligonucleotide overhang and a 3′ oligonucleotide overhang configured to hybridize to each other; wherein each of the more than one crRNAs comprises a repeat sequence and a spacer, wherein the spacer is configured to hybridize to a specific target nucleic acid of a plurality of target nucleic acids, and wherein the repeat sequence in each of the more than one crRNAs is preceded by a separator sequence. In other embodiments, the method of making a collection of engineered multiplex CRISPR arrays further comprises randomly hybridizing the more than one crRNAs to generate the collection of the engineered multiplex CRISPR arrays.

In some embodiments, the repeat sequences in the more than one crRNAs comprise Cas12a repeat sequence, a Cas13 repeat sequence, or both Cas12a and Cas13 repeat sequences. In certain embodiments, the Cas13 repeat sequence comprises a Cas13d repeat sequence and a Cas13b repeat sequence. In some embodiments, the collection of the engineered multiplex CRISPR arrays is capable of upregulating and downregulating the expression of the plurality of target nucleic acids simultaneously. In certain embodiments, the plurality of target nucleic acids comprises at least 4 different target nucleic acids.

In some embodiments, the average length of the crRNA is about 30 to about 70 nucleotides. In certain embodiments, the average length of the crRNA is about 50 nucleotides. In other embodiments, the spacer comprises an A or an T at the 3′ end.

In some embodiments, the separator sequence comprises an AT-rich linker sequence. In certain embodiments, the separator sequence is about 3 to about 8 nucleotides in length.

In some embodiments, the method further comprises identifying the collection of engineered multiplex CRISPR arrays having a desired length.

In additional embodiments, the method of making a collection of engineered multiplex CRISPR arrays further comprises inserting the collection of the engineered multiplex CRISPR arrays into a vector. In some embodiments, the vector comprises a eukaryotic expression vector.

In other embodiments, the method of making a collection of engineered multiplex CRISPR arrays further comprises delivering the collection of the engineered multiplex CRISPR arrays into host cells. In some embodiments, the host cells express the more than one Cas proteins.

In yet other embodiments, the method of making a collection of engineered multiplex CRISPR arrays further comprises screening for the collection of engineered multiplex CRISPR arrays with a desired phenotype. In some embodiments, the screening comprises isolating the host cells exhibiting the desired phenotype. In some embodiments, the screening further comprises sequencing the engineered multiplex CRISPR array expressed by the isolated host cells. In certain embodiments, the desired phenotype comprises controlled stem cell differentiation, controlled killing of tumor cells, and enhanced cell proliferation, increased T-cell activity level, and modified metabolic activity.

The present disclosure further provides a method for simultaneous upregulation of multiple endogenous genes, comprising contacting a host cell with the engineered multiplex CRISPR array described herein, wherein the more than one crRNAs comprise Cas12a repeat sequences and spacers configured to hybridize to a plurality of target nucleic acids.

In other embodiments, the present disclosure provides a method for simultaneous downregulation of multiple endogenous genes, comprising contacting a host cell with the engineered multiplex CRISPR array described herein, wherein the more than one crRNAs comprise Cas13 repeat sequences and spacers configured to hybridize to a plurality of target nucleic acids.

In further embodiments, the present disclosure provides a method for simultaneous upregulation and downregulation of multiple endogenous genes, comprising contacting a host cell with the engineered multiplex CRISPR array described herein, wherein the more than one crRNAs comprise both Cas12a and Cas13 repeat sequences and spacers configured to hybridize to a plurality of target nucleic acids.

In other embodiments of the present disclosure, the host cell expresses Cas12a proteins, Cas13 proteins, or both Cas12a proteins and Cas13 proteins.

### BRIEF DESCRIPTION OF THE DRAWINGS/FIGURES

FIGS. 1A-1M show that high spacer GC content negatively influences performance of the subsequent crRNA in a CRISPR array. FIG. 1A is an exemplary illustration of the CRISPR-Cas12a operon, which consists of a number of Cas genes and a CRISPR array (shown to scale) that can be transcribed as a single transcript. FIG. 1B is an exemplary illustration of a Cas12a array as it occurs naturally in bacteria, showing that each crRNA consists of a repeat and a spacer. Prior to crRNA processing, repeats contain a ˜14-18 nt fragment, which get excised by Cas12a and an unknown enzyme. This illustration highlights a gene fragment that separates each crRNA, which is denoted as a “CRISPR separator” (or “separator”). FIG. 1C is an exemplary illustration of the most commonly used CRISPR array design for use in mammalian cells. This design has omitted the separator because its function has not been known. FIG. 1D is an exemplary illustration of CRISPR arrays consisting of two crRNAs. The first crRNA contains a non-targeting spacer. The second cRNA's spacer targets the promoter of GFP, which is genomically integrated in HEK293T cells. FIG. 1E is an exemplary illustration of arrays and dCas12a-VPR which were transfected in HEK293T cells, and GFP fluorescence was analyzed as a measure of array performance. FIG. 1F shows the percentage of GFP positive cells for two spacers, along with their sequences, CGCCAAACGTGCCCTGACGGT (SEQ ID NO: 31) and CGCCAAACGTGCCCTGACGGG (SEQ ID NO: 32), showing that the arrays display hypersensitivity to the identity of the last base of the spacer. Replacing the last nucleotide from a T to a G leads to an almost complete failure of transfected cells to activate GFP expression. FIG. 1G shows the percent of GFP positive cells for generated arrays where the first crRNA contains one of 51 nonsense, non-targeting spacers with varying GC content. A strong negative correlation is seen between the GC content of the spacer and GFP fluorescence. (Each dot corresponds to one of the 51 CRISPR arrays and represents the average of triplicate experiments). Arrays were divided into three groups, Low GFP, Medium GFP, and High GFP, based on the level of GFP fluorescence they enabled. FIG. 1H shows the average GC content of a sliding 5-nt window calculated for the groups of FIG. 1G. This graph shows that the best-performing arrays were the ones where the spacer happened to have low GC content at its 3′ end. Some arrays showed unexpectedly high (as shown in FIG. 1I) or low (as shown in FIG. 1J) GFP activity for the GC content of their spacers. These arrays happened to contain particularly low (as shown in FIG. 1I) or high (as shown in FIG. 1J) GC content at the very 3′ end of their spacers. This suggested that the GC content of the last few bases is an important predictor of array performance in this experiment. FIG. 1K shows the predictive power (R²) of knowing the GC content of 3-nt regions of the spacer. As shown, simply knowing the GC content of the last 3 bases of the upstream spacer was more predictive of array performance compared to knowing the GC content of the entire spacer. Shaded regions in FIGS. 1G-1I represent standard error for those spacers. FIG. 1L shows the relationship between GC content of 51 non-targeting dummy spacers and the secondary structures they are predicted to form with the GFP-targeting gRNA (the larger the value on the y-axis, the more stable the predicted secondary structure). FIG. 1M shows that the predicted secondary structure formation is anticorrelated with performance of the GFP-targeting spacer, suggesting that strong secondary structures is what impedes array performance.

FIGS. 2A-2C show that CRISPR separators contain a region with conserved low GC content. FIG. 2A shows the GC content of 727 naturally occurring Cas12a spacers from 30 bacterial species. As shown, naturally occurring CRISPR-Cas12a arrays show no conspicuous depletion of spacers with high GC content. FIG. 2B is an exemplary illustration of a portion of Cas12a CRISPR arrays, with graphs showing the sliding average GC content for 727 naturally occurring Cas12a spacers (left graph) and 79 Cas12a separators (right graph). The naturally occurring spacers do not show low GC content at their 3′ ends. However, the separator sequences of these crRNAs have low GC content. This is seen also in a multiple-sequence alignment of separator sequences (as shown in FIG. 2C). This suggests that the purpose of the CRISPR separator is to act as an insulator between adjacent crRNAs in a CRISPR-Cas12a array.

FIGS. 3A-3I show that the introduction of a short, artificial separator between crRNAs improves performance of Cas12a arrays in human cells. FIGS. 3A-3B are exemplary illustrations of Cas12a arrays (FIG. 3A) and an artificial separator (FIG. 3B). Fifteen variants of a 2-crRNA array were tested. Each array contained an artificial separator (G, T, AT, AAT, or AAAT), and the GC content of the spacer was 30%, 50%, or 70%. FIG. 3C shows the percentage of GFP positive cells for spacers with GC content of 30%, 50%, and 70%. In each case, array performance was improved the more AT nucleotides were added. FIG. 3D is an exemplary illustration of a 7-cRNA array that was designed to activate seven endogenous genes in HEK293T cells and either included or omitted the artificial AAAT separator between each crRNA. FIG. 3E shows relative RNA level compared to control gene RPL13A for several target genes. For all target genes, the AAAT separator improved target gene activation level, as measured by RT-qPCR. The improvement was consistent (1.1 to 8.0 fold) for all seven genes (as shown in FIG. 3F). FIG. 3G shows median GFP fluorescence showing that the improvement was also seen on the protein level for the target gene GFP, as measured by GFP fluorescence and percent GFP-positive cells. FIG. 3H shows that short, artificial separators derived from multiple bacterial species can rescue poor GFP activation caused by a non-permissive non-targeting dummy spacer upstream of the targeting spacer in a CRISPR array. FIG. 3I shows that the enhanced Cas12a protein from Acidaminococcus species is also sensitive to GC content of an upstream non-targeting dummy spacer and that its performance can be rescued using a TTTT synSeparator derived from its natural separator.

FIG. 4 shows a multiple-sequence alignment of 79 separators from 30 bacterial species. A partial sequence alignment is shown in FIG. 2C.

FIG. 5 shows an exemplary illustration of a Cas12a CRISPR array.

FIG. 6 is an exemplary, non-limiting illustration of the major steps of the method of making a collection of engineered multiplex CRISPR arrays provided herein.

FIGS. 7A-7D show exemplary designs of hybrid engineered multiplex CRISPR arrays described herein. FIGS. 7A and 7B show examples of hybrid engineered multiplex CRISPR arrays as described herein. FIG. 7C shows an example of a CRISPR Cas12a/Cas13d hybrid array consisting of two Cas13d gRNAs whose spacers target GFP mRNA for destruction and GFP downregulation, and one Cas12a gRNA whose spacer targets the CD9 gene for upregulation. This array was transfected into HEK293T cells constitutively expressing GFP, and was co-transfected with the dCas12a-miniVPR activator and/or Cas13d. The plot represents flow cytometry data of cells stained with an APC-conjugated CD9-targeting antibody, and shows that cells transfected with both Cas proteins simultaneously downregulate GFP and upregulate CD9 compared to non-transfected control cells. FIG. 7D shows 5 different designs of CRISPR Cas12a/Cas13d hybrid arrays, all of which demonstrate simultaneous upregulation of the Cas12a target gene CD9 and downregulation of the Cas13d target gene GFP, as demonstrated by flow cytometry readout of cells stained with an APC-conjugated CD9-targeting antibody.

### DETAILED DESCRIPTION OF THE DISCLOSURE

The present disclosure provides an optimized design of CRISPR arrays that enable simultaneous, multi-mode gene expression regulation (e.g., simultaneous upregulation and down regulation of multiple target genes). In some embodiments, the present disclosure demonstrates that incorporating a short, AT-rich separator sequence between each CRISPR-RNA (crRNA) in a CRISPR array improves the performance of the engineered multiplex CRISPR array. In some embodiments, the present disclosure provides a novel design for a hybrid CRISPR array comprising crRNAs for multiple Cas proteins, such as, but not limited to, Cas12a and Cas13. In some embodiments, the hybrid engineered multiplex CRISPR arrays enable simultaneous upregulation and downregulation of multiple target genes using a single CRISPR array.

### I. Definitions

As used herein, the singular forms “a,” “an,” and “the” include both singular and plural referents unless the context clearly dictates otherwise.

The term “optional” or “optionally” means that the subsequent described event, circumstance or substituent may or may not occur, and that the description includes instances where the event or circumstance occurs and instances where it does not.

Where a range of values is provided, it is understood that each intervening value, to the tenth of the unit of the lower limit unless the context clearly dictates otherwise, between the upper and lower limit of that range and any other stated or intervening value in that stated range, is encompassed within the disclosure. The upper and lower limits of these smaller ranges may independently be included in the smaller ranges, and are also encompassed within the disclosure, subject to any specifically excluded limit in the stated range. Where the stated range includes one or both of the limits, ranges excluding either or both of those included limits are also included in the disclosure.

Certain ranges are presented herein with numerical values being preceded by the term “about.” The term “about” is used herein to provide literal support for the exact number that it precedes, as well as a number that is near to or approximately the number that the term precedes, such as variations of +/−10% or less, +/−1-5% or less, +/−1% or less, and +/−0.1% or less from the specified value. In determining whether a number is near to or approximately a specifically recited number, the near or approximating unrecited number may be a number which, in the context in which it is presented, provides the substantial equivalent of the specifically recited number.

The terms “subject” and “individual” are used interchangeably herein to refer to a vertebrate, preferably a mammal, more preferably a human. In some cases, a subject is a patient. Mammals include, but are not limited to, murines, simians, humans, farm animals, sport animals, and pets. Tissues, cells and their progeny of a biological entity obtained in vivo or cultured in vitro are also encompassed.

It is appreciated that certain features of the disclosure, which are, for clarity, described in the context of separate embodiments, may also be provided in combination in a single embodiment. Conversely, various features of the disclosure, which are, for brevity, described in the context of a single embodiment, may also be provided separately or in any suitable sub-combination. All combinations of the embodiments pertaining to the disclosure are specifically embraced by the present disclosure and are disclosed herein just as if each and every combination was individually and explicitly disclosed. In addition, all sub-combinations of the various embodiments and elements thereof are also specifically embraced by the present disclosure and are disclosed herein just as if each and every such sub-combination was individually and explicitly disclosed herein.

All publications, published patent documents, and patent applications cited herein are hereby incorporated by reference to the same extent as though each individual publication, published patent document, or patent application was specifically and individually indicated as being incorporated by reference.

### II. Compositions

### Engineered Multiplex CRISPR Array

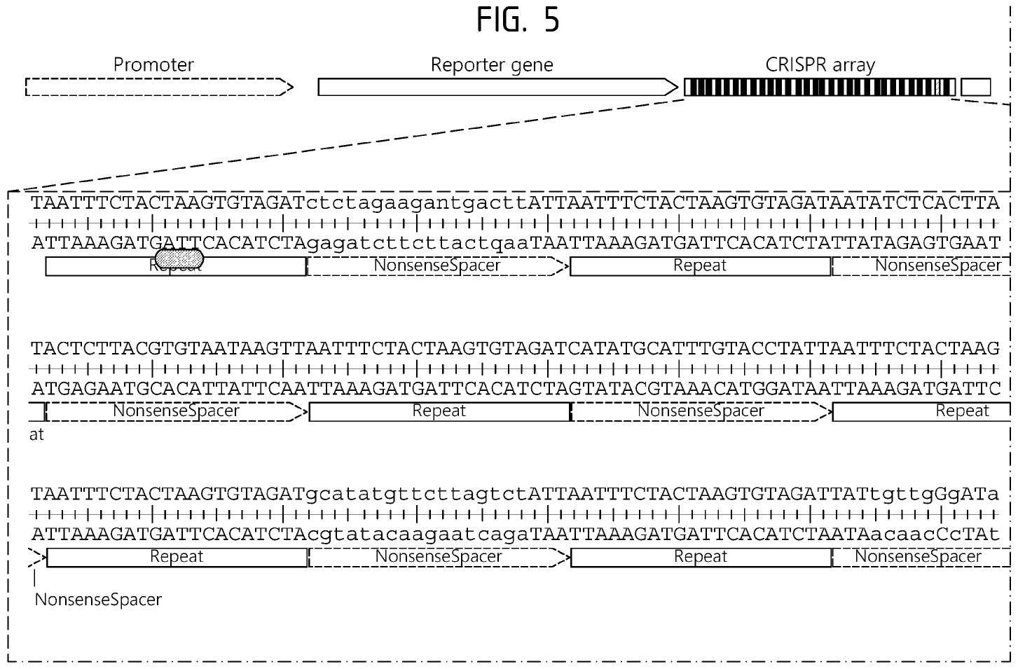

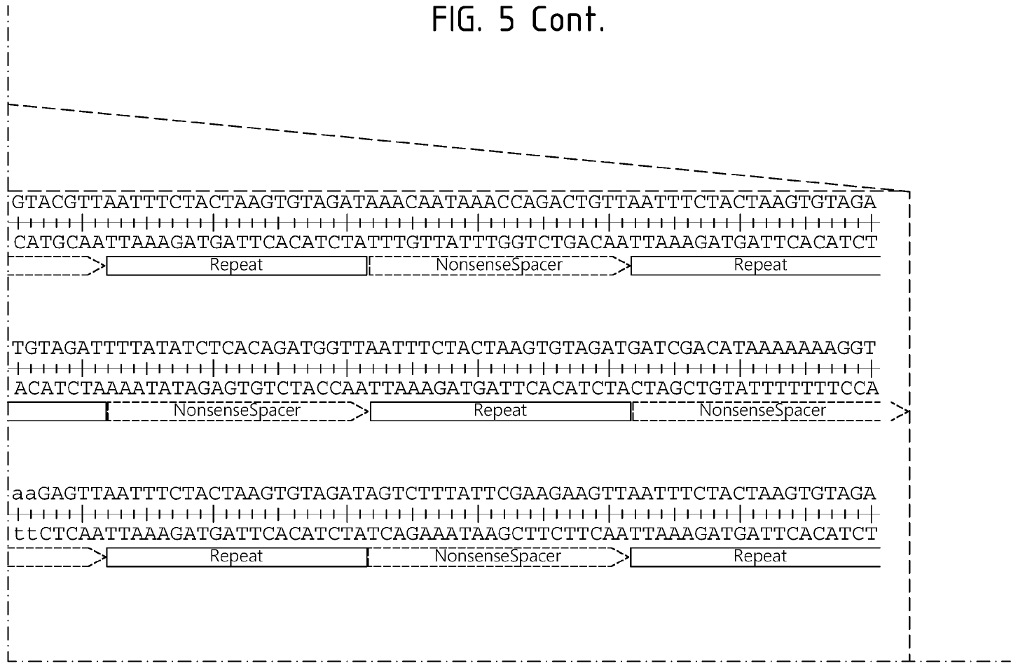

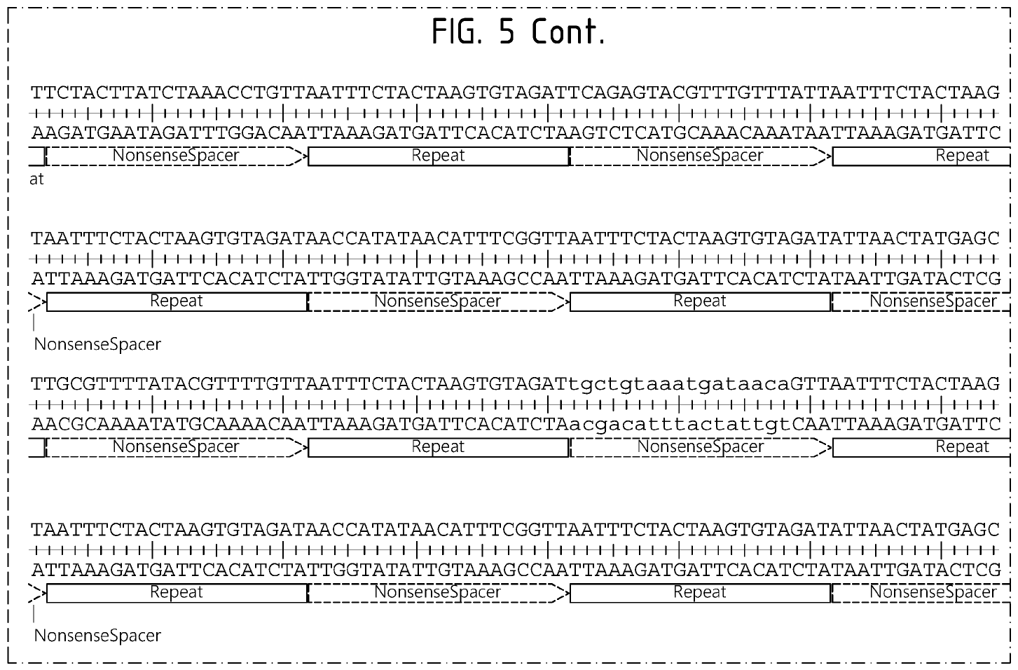

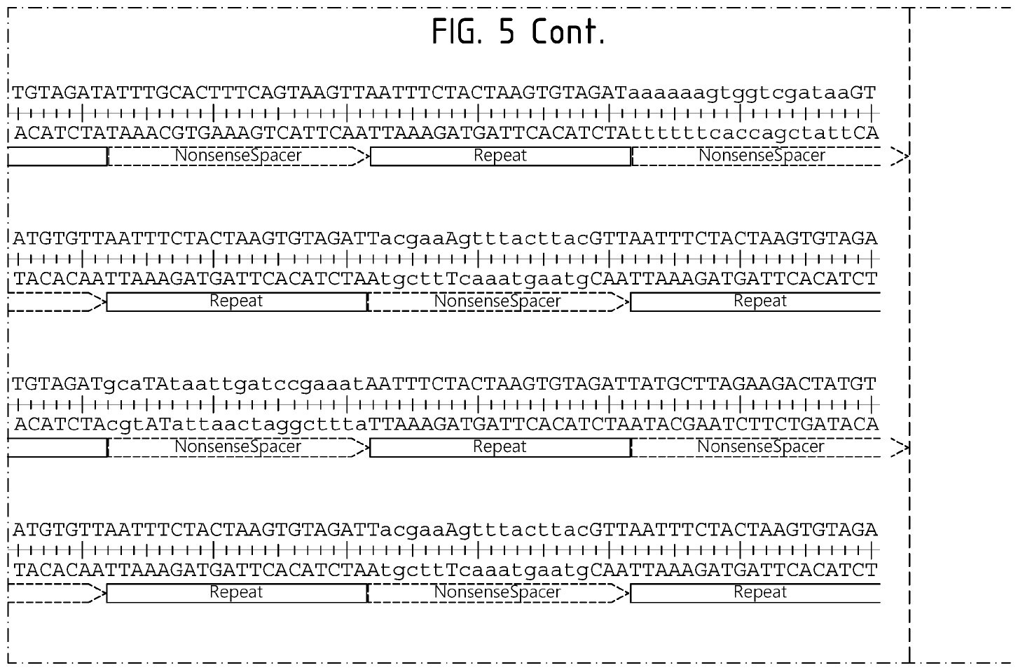

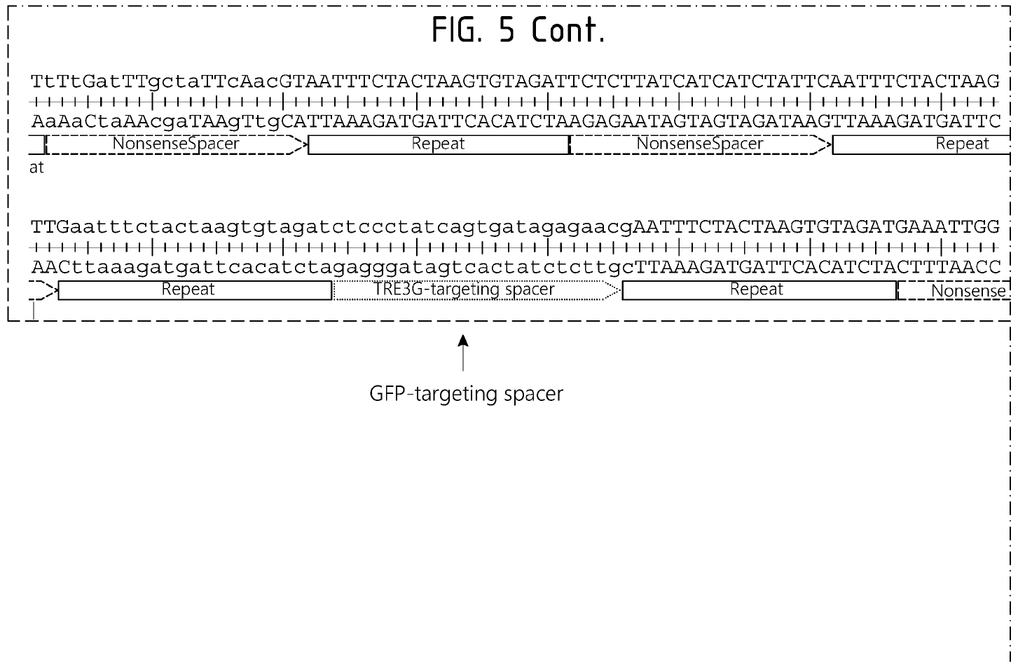

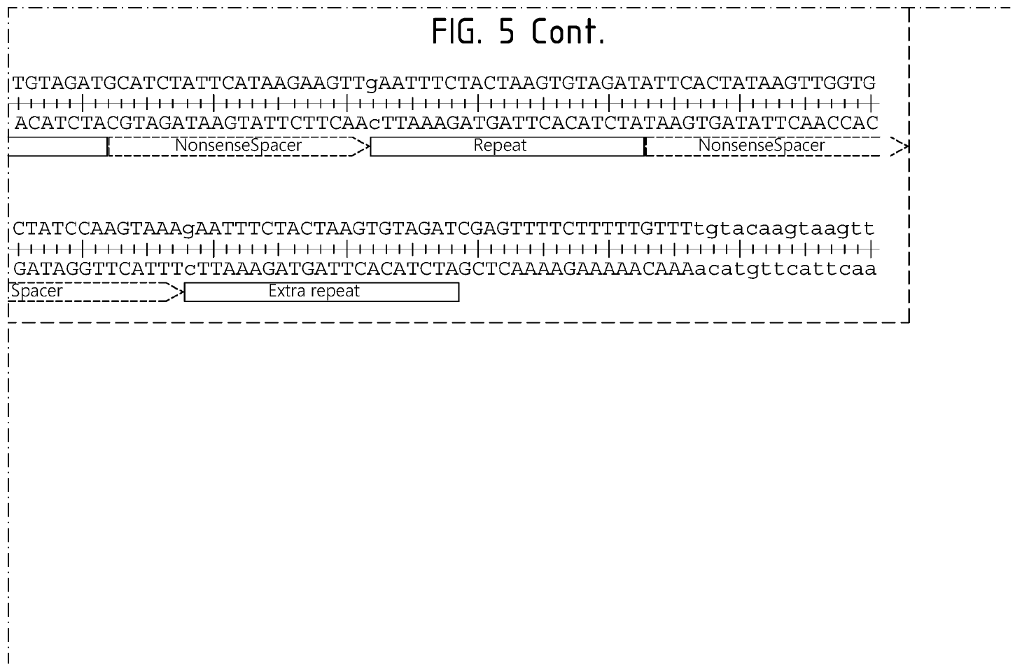

In some embodiments, the present disclosure provides an engineered multiplex Clustered Regularly Interspaced Short Palindromic Repeat (CRISPR) array. In some embodiments, the engineered multiplex CRISPR array comprises more than one CRISPR RNAs (crRNAs). In some embodiments, the more than one crRNAs are arranged in tandem, i.e., located immediately adjacent to one another on a CRISPR array. In some embodiments, each of the crRNAs comprises a repeat sequence and a spacer. In some embodiments, the repeat sequence in the each of the crRNAs is immediately preceded by a separator sequence. An exemplary engineered multiplex CRISPR array is illustrated in FIG. 5. Each of the components is described herein.

The engineered multiplex CRISPR array provided herein can comprise any number of crRNAs as needed. In some embodiments, the engineered multiplex CRISPR array provided herein comprises 2-10 crRNAs. In some embodiments, the engineered multiplex CRISPR array provided herein comprises 4 or more crRNAs. In some embodiments, the engineered multiplex CRISPR array provided herein comprises 5 or more crRNAs. In some embodiments, the engineered multiplex CRISPR array provided herein comprises 6 or more crRNAs. In some embodiments, the engineered multiplex CRISPR array provided herein comprises 7 or more crRNAs. In some embodiments, the engineered multiplex CRISPR array provided herein comprises 8 or more crRNAs. In some embodiments, the engineered multiplex CRISPR array provided herein comprises 9 or more crRNAs. In some embodiments, the engineered multiplex CRISPR array provided herein comprises 10 or more crRNAs. In other embodiments, the engineered multiplex CRISPR array provided herein comprises more than 10 crRNAs. In some embodiments, the engineered multiplex CRISPR array provided herein comprises about 10 to about 100 crRNAs. In other embodiments, the engineered multiplex CRISPR array provided herein comprises more than about 100 crRNAs.

As used herein, the term “CRISPR RNA” or “crRNA” refers to a guide RNA (gRNA) molecule having a synthetic sequence and typically comprising two sequence components: a spacer sequence and a gRNA scaffold sequence (also called a “repeat sequence”). These two sequence components can be in a single RNA molecule or in a double-RNA molecule configuration (also known as a duplex guide RNA that comprises both a CRISPR RNA (crRNA) and a trans-activating crRNA (tracrRNA)). In some instances, a gRNA can have a crRNA component only (without a tracrRNA), for example, gRNAs that work with Cas12a (also known as Cpf1)). In some embodiments, a CRISPR associate protein as described herein may utilize a guide nucleic acid comprising DNA, RNA or a combination of DNA and RNA. The term “guide nucleic acid” is inclusive, referring both to double-molecule guides and to single-molecule guides.

As used herein, a CRISPR associated (“Cas”) nuclease refers to a protein encoded by a gene generally coupled, associated or close to or in the vicinity of flanking CRISPR loci, and further capable of introducing a double strand break into a target nucleic acid sequence (e.g., RNA or DNA). The terms “Cas nuclease” and “Cas protein” are used interchangeably herein. In some embodiments, a Cas protein is guided by a guide polynucleotide to recognize and introduce a double strand break at a specific target site into the genome of a cell. Upon recognition of a target sequence by a CRISPR RNA (also called crRNA), a Cas protein unwinds the DNA duplex in close proximity of the target sequence and cleaves both DNA strands or a target RNA strand, but only if the correct protospacer-adjacent motif (PAM) is approximately oriented at the 3′ end of the target sequence.

In some embodiments, the Cas protein is a Cas12a. Cas12a is an RNA-programmable DNA endonuclease. Cas12a has intrinsic RNase activity that allows processing of its own crRNA array, enabling multigene editing from a single RNA transcript. Typically, a Cas12a nuclease binds double-stranded DNAs (dsDNA). In some embodiments, the Cas12a endonuclease is from Lachnospiraceae bacterium, Acidaminococcus sp. or Francisella tularensis subsp. novicida. One exemplary illustration of a Cas12a CRISPR array is shown in FIG. 5.

In other embodiments, the Cas protein encompassed herein comprises Cas13 nucleases. The diverse Cas13 family contains at least four known subtypes, including Cas13a (formerly C2c2), Cas13b, Cas13c, and Cas13d. Typically, Cas13 proteins use a ˜64-nt guide RNA to encode target specificity. The Cas13 protein complexes with the crRNA (i.e., a Cas13 repeat sequence) via recognition of a short hairpin in the crRNA, and target specificity is encoded by a 28 to 30 nucleotides long spacer that is complementary to the target region. In addition to programmable RNase activity, all Cas13s exhibit collateral activity after recognition and cleavage of a target transcript, leading to non-specific degradation of any nearby transcripts regardless of complementarity to the spacer. In some embodiments, a Cas13 protein can programmatically bind and cleave endogenous RNA. In certain embodiments, the Cas13 nuclease comprises a Cas13d nuclease and/or a Cas13b nuclease. In some embodiments, the Cas13b endonuclease is from Porphyromonas gulae or Prevotella sp. In some embodiments, the Cas13d endonuclease is from Ruminococcus flavefaciens.

In certain embodiments, the Cas protein is a deactivated Cas protein. As used herein, a “deactivated Cas protein” (dCas) refers to a nuclease comprising a domain that retains the ability to bind its target nucleic acid but has a diminished, or eliminated, ability to cleave a nucleic acid molecule, as compared to a control nuclease. In certain embodiments, a catalytically inactive nuclease is derived from a “wild type” Cas protein. As used herein, a “wild type” nuclease refers to a naturally-occurring nuclease. In some embodiments, the catalytically inactive nuclease is a catalytically inactive Cas12a. In some embodiments, the catalytically inactive Cas12a produces a nick in the targeting strand. In some embodiments, the catalytically inactive Cas12a produces a nick in the nontargeting strand. In some embodiments, the catalytically inactive Cpfl, known as dead Cas12a (dCas12a), lacks all DNase activity. In some embodiments, the catalytically inactive Cas12a is a dCas12a endonuclease from Acidaminococcus sp. BV3L6 or Lachnospiraceae bacterium or Francisella tularensis subsp. novicida.

In some embodiments, the average length of each of the one or more crRNAs is about 20 to about 200 nucleotides long. In some embodiments, the average length of each of the one or more crRNAs is about 30 to about 100 nucleotides long. In some embodiments, the average length of each of the one or more crRNAs is about 30 to about 70 nucleotides long. In some embodiments, the average length of each of the one or more crRNAs is about 35 to about 65 nucleotides long. In some embodiments, the average length of each of the one or more crRNAs is about 40 to about 60 nucleotides long. In some embodiments, the average length of each of the one or more crRNAs is about 45 to about 55 nucleotides long. In certain embodiments, the average length of the crRNA is about 50 nucleotides long.

In some embodiments, each crRNA comprises a repeat sequence. In some embodiments, the repeat sequence is about 8-30 nucleotides long. In some embodiments, the repeat sequence is about 10-25 nucleotides long. In some embodiments, the repeat sequence is about 12-22 nucleotides long. In some embodiments, the repeat sequence is about 14-20 nucleotides long. In some embodiments, the repeat sequence is about 14-18 nucleotides long.

In some embodiments, the repeat sequence is identical for all crRNAs in the engineered multiplex CRISPR array. In other embodiments, the repeat sequences are different for all crRNAs in the engineered multiplex CRISPR array. In some embodiments, the engineered multiplex CRISPR arrays comprising different repeat sequences are called hybrid CRISPR arrays, or hybrid arrays for short.

The engineered multiplex CRISPR array provided herein can be used with any natural or modified versions of the CRISPR/Cas system, such as the first generation of dCas9-based CRISPR interference (CRISPRi) and CRISPR activation (CRISPRa) (CRISPRi/a, collectively). The various CRISPR/Cas system can be used to up- and downregulate endogenous genes. The currently available systems of methods have major limitations. For example, the users must choose whether to upregulate or downregulate genes. However, the users cannot choose to do both at the same time, unless they use two separate plasmids to express the guide-RNAs meant for upregulation or downregulation, respectively. However, using multiple plasmids is problematic as it is not possible to ensure that every cell takes up both plasmids, especially not at desired stoichiometric ratios. For at least this reason, the novel compositions and methods described herein provides a new generation of CRISPRi/a, collectively, which expands the capabilities in terms of throughput, multiplexing, and modes of control on the CRISPRi/a side.

In some embodiments, at least a portion of the more than one crRNAs comprise a Cas12a repeat sequence. An example of a naturally occurring Cas12a repeat sequence from Lachnospiraceae bacterium comprises AATTTCTACTAAGTGTAGAT (SEQ ID NO: 1). Another example of a naturally occurring Cas12a repeat sequence from Acidaminococcus sp. repeat sequence comprises AATTTCTACTCTTGTAGAT (SEQ ID NO: 112). The engineered multiplex CRISPR arrays provided herein can also be used with other subclasses of Cas12. In some embodiments, subclasses of Cas12, such as, without being limited to, Cas12a, Cas12b, Cas12c, Cas12d, Cas12e, Cas12f, Cas12g, Cas12h, and Cas12i, are also contemplated herein. Accordingly, the naturally occurring and/or artificial repeat sequences for the subclasses of Cas12 are also encompassed by the present disclosure. Further, the engineered multiplex CRISPR arrays provided herein can be compatible with other known or new Cas12 orthologs, which are also encompassed herein.

In other embodiments, at least a portion of the more than one crRNAs comprise a Cas13 repeat sequence. An example of a naturally occurring Cas13 repeat sequence comprises CAAGTAAACCCCTACCAACTGGTCGGGGTTTGAAAC (SEQ ID NO: 2). In some embodiments, at least a portion of the more than one crRNAs comprise a Cas12a repeat sequence and at least a portion of the more than one crRNAs comprise a Cas13 repeat sequence. In some embodiments, the Cas13 protein comprises a Cas13d protein and a Cas13b protein.

In some embodiments, at least a portion of the more than one crRNAs comprise a Cas12a repeat sequence and at least a portion of the more than one crRNAs comprise a Cas13 repeat sequence. In certain embodiments, at least a portion of the more than one crRNAs comprise a Cas12a repeat sequence and at least a portion of the more than one crRNAs comprise a Cas13b repeat sequence. In other embodiments, at least a portion of the more than one crRNAs comprise a Cas12a repeat sequence and at least a portion of the more than one crRNAs comprise a Cas13d repeat sequence.

In some embodiments, the crRNAs comprising different Cas proteins are presented in the same construct. These hybrid CRISPR arrays provided herein, for example, the hybrid CRISPR arrays encoding both Cas12a and Cas13 (e.g., Cas13d and/or Cas13b) crRNAs, solve the limitations of currently available methods mentioned above. Specifically, in some embodiments, the hybrid engineered multiplex CRISPR array provided herein enables simultaneous upregulation and downregulation of multiple genes using a single construct in the same cell, such that every cell that takes up this construct will up- and down-regulate the same set of genes as all other cells.

In some embodiments, each crRNA further comprises a spacer. Thus, in some embodiments, each of the more than one crRNA in the engineered multiplex CRISPR array comprises a repeat sequence and a spacer. In some embodiments, the engineered multiplex CRISPR array provided herein comprises spacers configured to hybridize to a plurality of target nucleic acids. Specifically, in some embodiments, the engineered multiplex CRISPR array provided herein comprises spacers comprising sequences that are complementary to their respective target nucleic acid sequences. The complementarity can be partial complementarity or complete (e.g., perfect) complementarity.

The terms “complementary” and “complementarity” are used as they are in the art and refer to the natural binding of nucleic acid sequences by base pairing. The complementarity of two polynucleotide strands is achieved by distinct interactions between nucleobases: adenine (A), thymine (T) (uracil (U) in RNA), guanine (G), and cytosine (C). Adenine and guanine are purines, while thymine, cytosine, and uracil are pyrimidines. Both types of molecules complement each other and can only base pair with the opposing type of nucleobase by hydrogen bonding. For example, an adenine can only be efficiently paired with a thymine (A=T) or a uracil (A=U), and a guanine can only be efficiently paired with a cytosine (GC). The base complement A=T or A=U shares two hydrogen bonds, while the base pair GC shares three hydrogen bonds. The two complementary strands are oriented in opposite directions, and they are said to be antiparallel. For another example, the sequence 5′-A-G-T 3′ binds to the complementary sequence 3′-T-C-A-5′. The degree of complementarity between two strands may vary from complete (or perfect) complementarity to no complementarity. The degree of complementarity between polynucleotide strands has significant effects on the efficiency and strength of the hybridization between the nucleic acid strands. In some embodiments, the polynucleotide probes provided herein comprise two perfectly complementary strands of polynucleotides.

As used herein, the term “perfectly complementary” means that two strands of a double-stranded nucleic acid are complementary to one another at 100% of the bases, with no overhangs on either end of either strand. For example, two polynucleotides are perfectly complementary to one another when both strands are the same length, e.g. 100 bp in length, and each base in one strand is complementary to a corresponding base in the “opposite” strand, such that there are no overhangs on either the 5′ or 3′ end.

In some embodiments, each spacer is configured to hybridize to a different target nucleic acid. In other embodiments, at least a portion of the spacers in a CRISPR array provided herein are configured to hybridize to the same target nucleic acid, while other spacers are configured to hybridize to different target nucleic acids.

In some embodiments, the spacer is about 10 to about 40 nucleotides long. In some embodiments, the spacer is about 20 to about 35 nucleotides long. In some embodiments, the spacer is about 10 to about 30 nucleotides long. In some embodiments, the spacer is about 15 to about 25 nucleotides long. In some embodiments, the spacer is about 18 to about 28 nucleotides long. In certain embodiments, the spacer is about 20 nucleotides long. In other embodiments, the spacer is about 22 nucleotides long. In yet other embodiments, the spacer is about 24 nucleotides long. In some exemplary embodiments, a spacer for a Cas12 protein is about 15-23 nucleotides long. In other exemplary embodiments, a spacer for a Cas13 protein is about 23-30 nucleotides long.

In some embodiments, a spacer sequence provided herein is not naturally occurring. In some embodiments, the spacer has a GC content of about 90% or lower. In some embodiments, the spacer has a GC content of about 80% or lower. In some embodiments, the spacer has a GC content of about 20%-80%. In some embodiments, the spacer has a GC content of about 30% to about 70%. In some embodiments, the spacer has a GC content of about 40% to about 60%. In other embodiments, the spacer has a GC content of about 50%.

In certain embodiments, the present disclosure demonstrates that particularly permissive spacers, i.e., spacers that tend to allow the processing of the subsequent crRNA, have a GC content that decreases toward the 3′ end of the spacer. In some embodiments, the spacers comprise more than 2 As and/or Ts (A/T) in the last 5 bases at the 3′ end. In some embodiments, the spacers comprise more than 3 A/T in the last 5 bases at the 3′ end. In some embodiments, the spacers comprise more than 4 A/T in the last 5 bases at the 3′ end. In some embodiments, the spacers of the present disclosure comprise all As/Ts in the last 5 bases at the 3′ end. In some embodiments, the spacers of the present disclosure comprise all As/Ts in the last 3 bases at the 3′ end. In some embodiments, the spacers of the present disclosure comprise an A/T at the 3′ end. In other embodiments, the present disclosure demonstrates that particularly non-permissive spacers have GC content higher toward the 3′ end of the spacer. In some embodiments, the spacer has a relatively high average GC content, it still allows efficient performance of the subsequent crRNA if the GC content is low in the last 3-5 bases at its 3′ end. Non-limiting exemplary sequences for spacers used herein are provided in Table 2.

In some embodiments, the present disclosure demonstrates that the spacers in a CRISPR array interfere with the performance of the crRNAs directly downstream of them. In certain embodiments, the higher the GC content of a spacer is, the more it negatively interferes with the function of the subsequent crRNA. Thus, in some embodiments, an AT-rich separator sequence is inserted between each crRNA in the CRISPR arrays provided herein. Surprisingly, it is found that the inclusion of such a separator improves the performance of the engineered multiplex CRISPR array (e.g., a Cas12a CRISPR array) and allows more effective CRISPR-upregulation (e.g., activation) of target nucleic acids in host cells. In some embodiments, the separator sequence acts as an insulator that reduces interference between adjacent crRNAs in an array. In some embodiments, the performance of the engineered multiplex CRISPR array, such as a Cas12a CRISPR array, is improved by the addition of a separator sequence between crRNAs. Furthermore, the present disclosure demonstrates that the inclusion of an artificial separator sequence disclosed herein removes the disruptive effects of GC content of the upstream spacer.

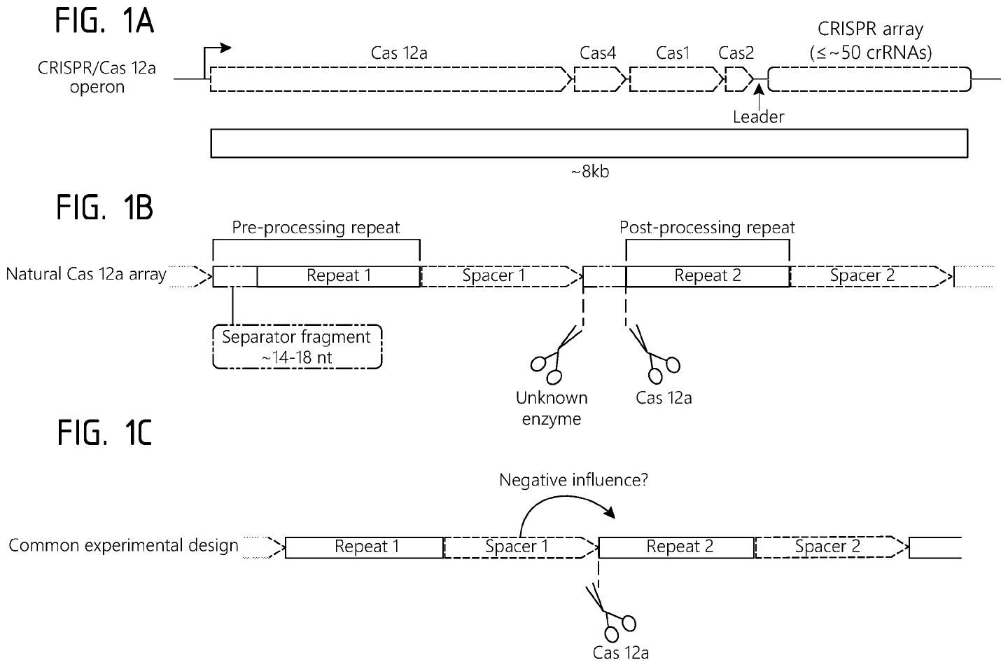

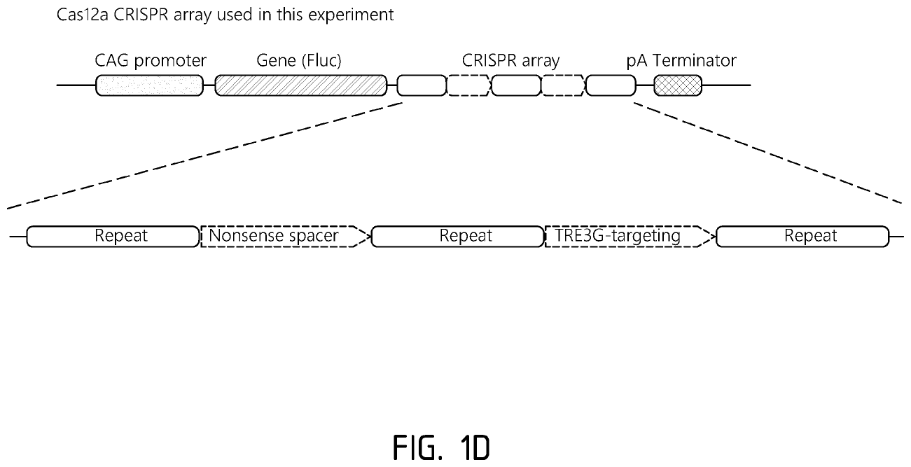

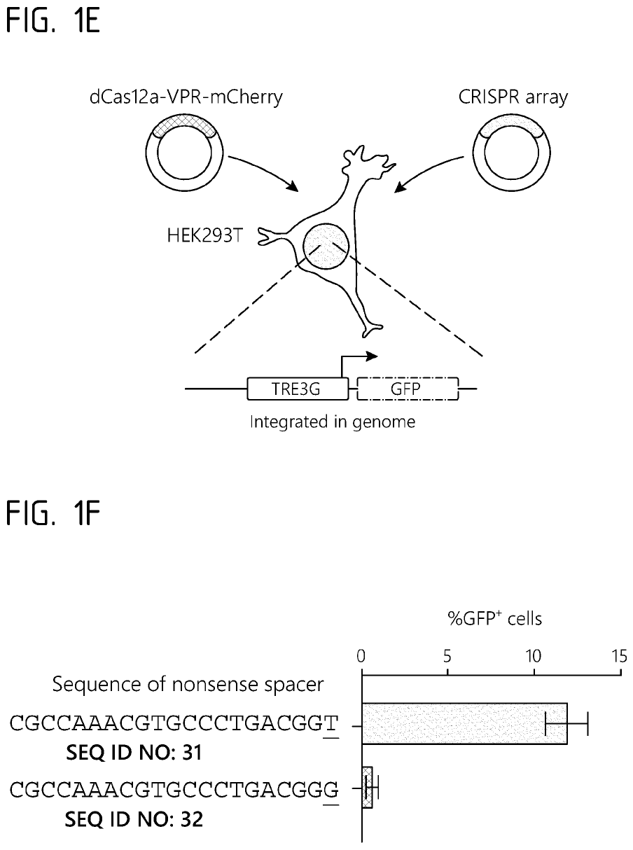

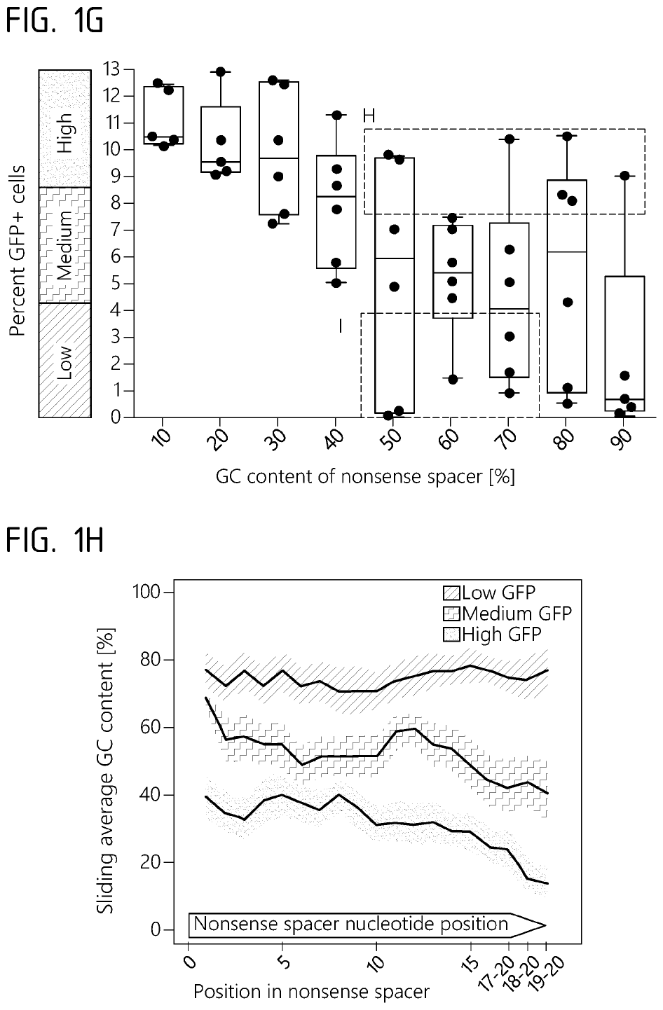

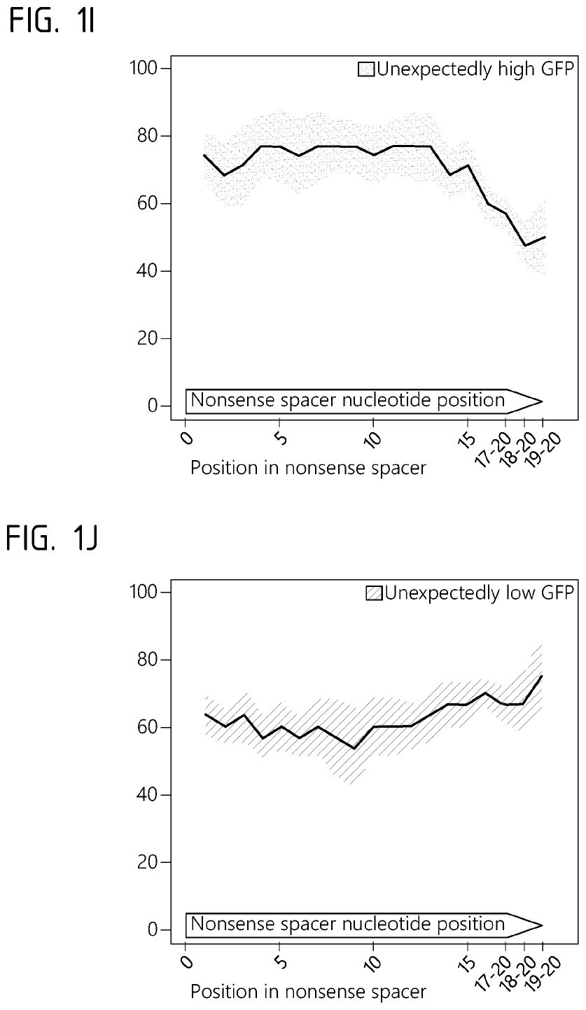

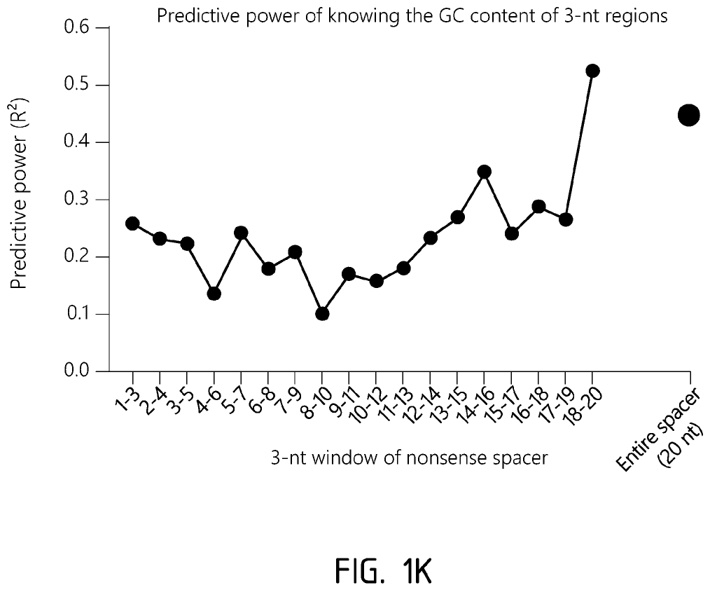

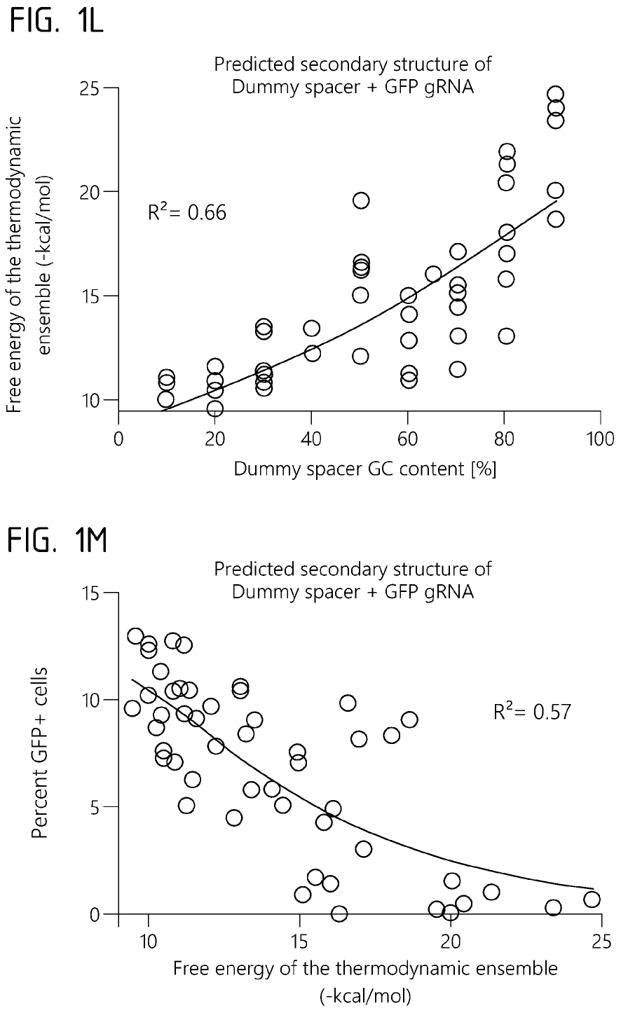

In some embodiments, the repeat sequence in the crRNAs is immediately preceded by a separator sequence. FIG. 1C is an exemplary illustration of a separator sequence. Traditionally, this fragment is not strictly required. Typically, a Cas12a nuclease cannot excise the separator on its own. Therefore, a separator sequence is often omitted when Cas12a arrays have been experimentally expressed in eukaryotic cells, such as mammalian cells. In contrast, the CRISPR arrays provided herein comprise a separator sequence preceding the repeat sequence in each crRNA. In some embodiments, the present disclosure demonstrates that the separator could serve to insulate crRNAs from the negative influence of secondary structure that might form in spacers. In some embodiments, the crRNA including the preceding separator sequence is referred to as a pre-crRNA. For instance, in the natural bacterial setting, the repeat sequence typically includes a short (e.g., about 16-about 18 nt) fragment which is subsequently excised and discarded during CRISPR processing and maturation. The resulting final crRNA typically consists of a post-processing repeat and spacer. An exemplary illustration is provided in FIG. 1B. In some embodiments, the excised repeat fragment, which is denoted as a CRISPR separator or a separator, is cleaved in its 3′ end by a Cas protein (e.g., Cas12a), and in its 5′ end by another enzyme (FIG. 1B).

In some embodiments, the separator sequence comprises an AT-rich sequence. In some embodiments, the separator sequence has an AT content of more than about 40%. In other embodiments, the separator sequence has an AT content of more than about 50%. In some embodiments, the separator sequence has an AT content of more than about 60%. In other embodiments, the separator sequence has an AT content of more than about 70%. In some embodiments, the separator sequence has an AT content of more than about 80%. In other embodiments, the separator sequence has an AT content of more than about 90%. In certain embodiments, the separator sequence has an AT content of about 100%.

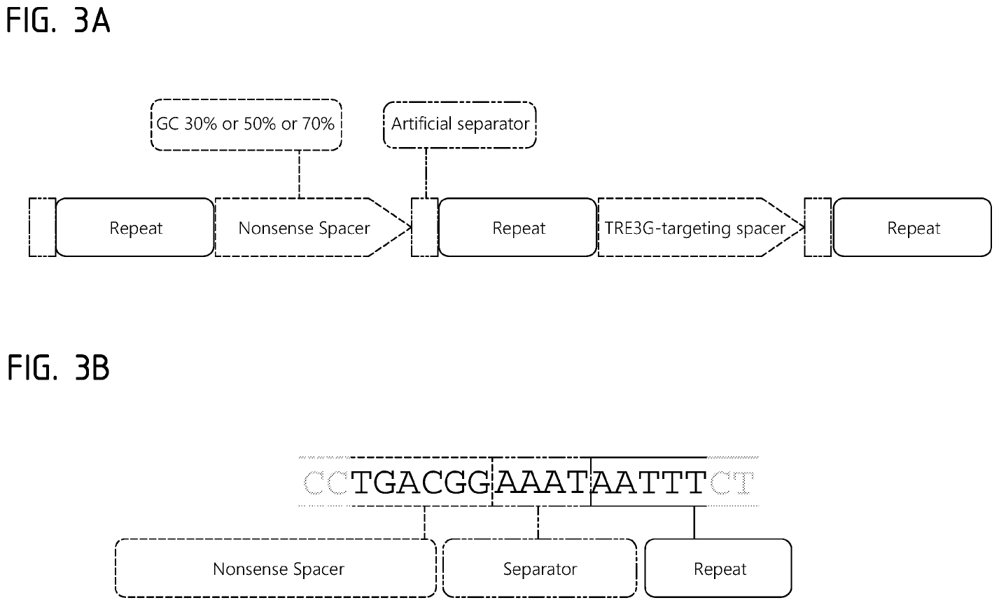

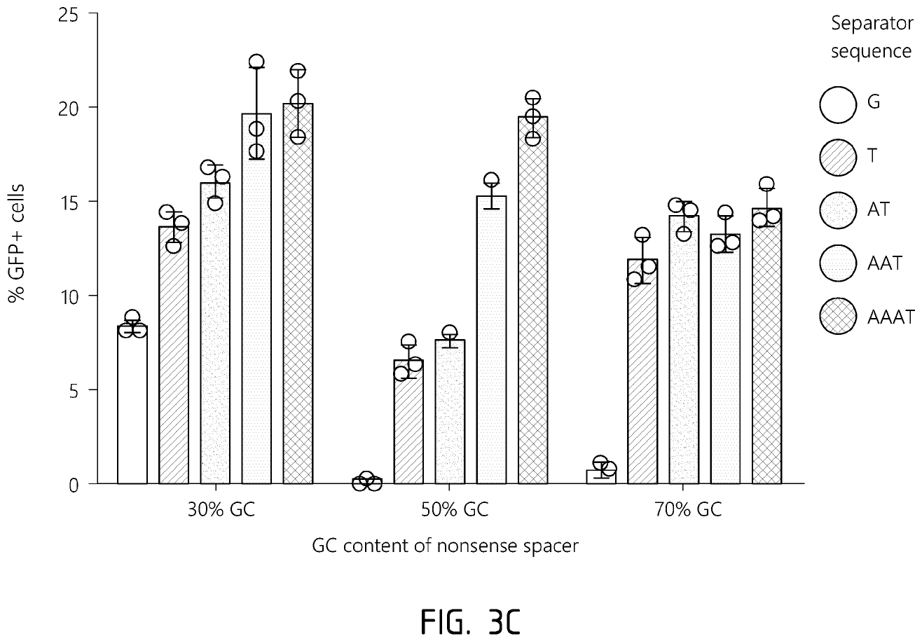

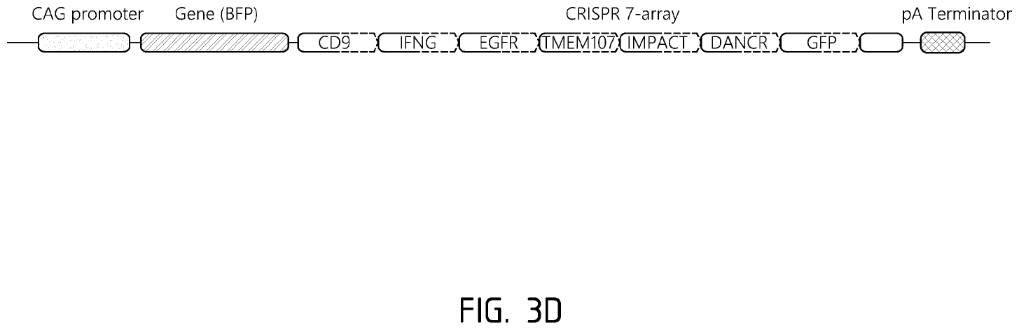

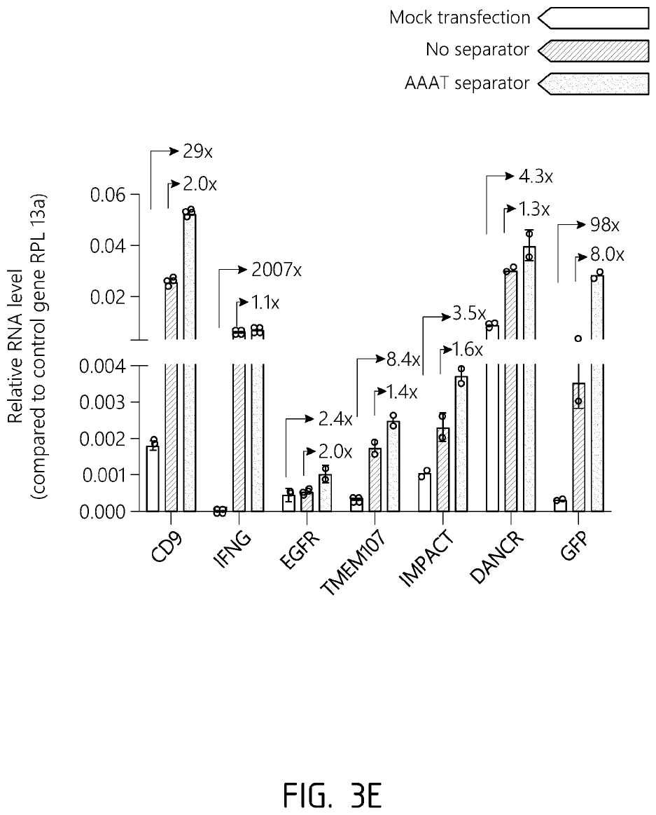

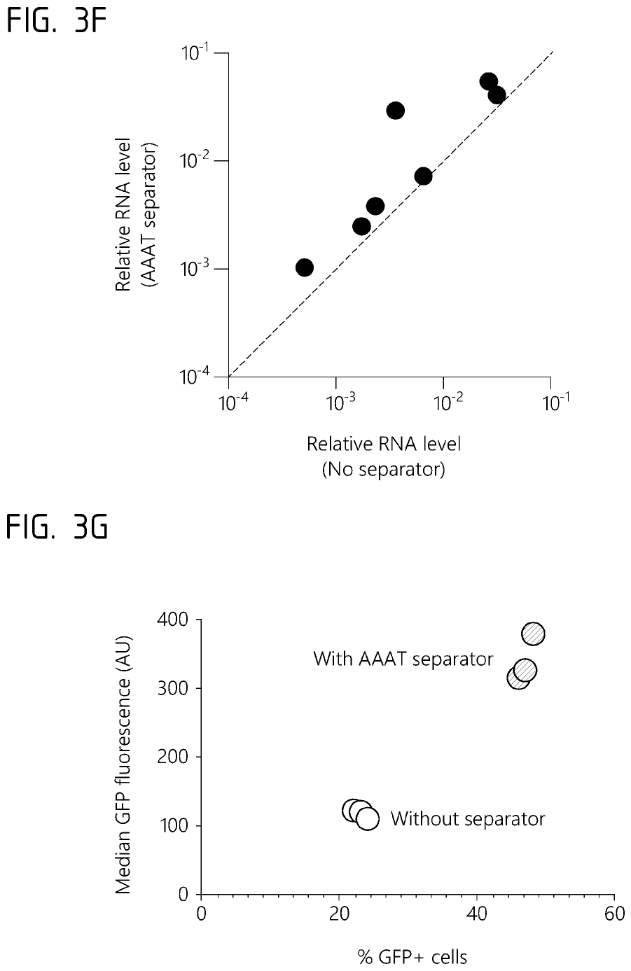

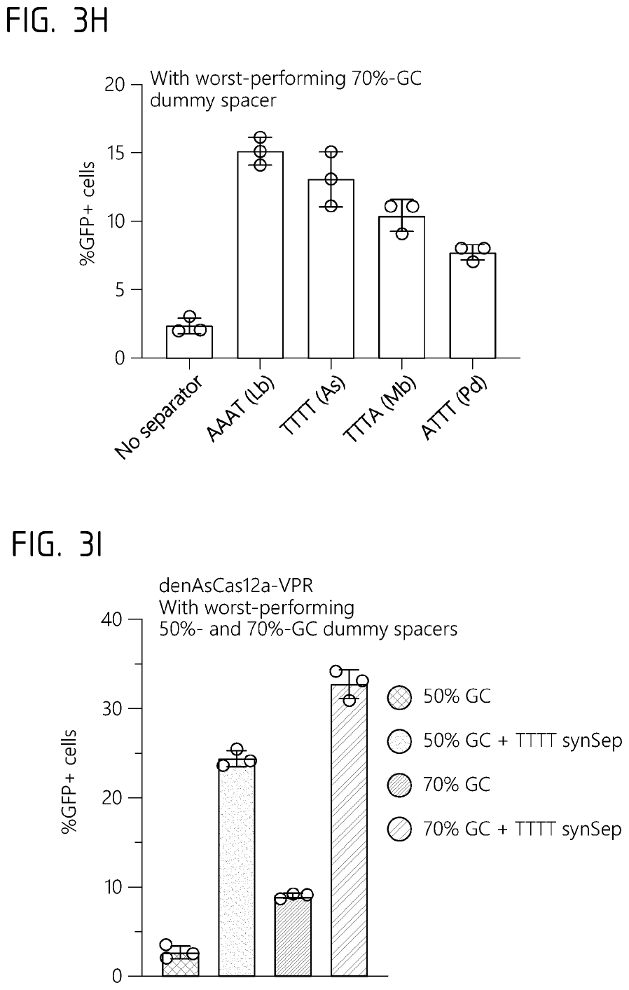

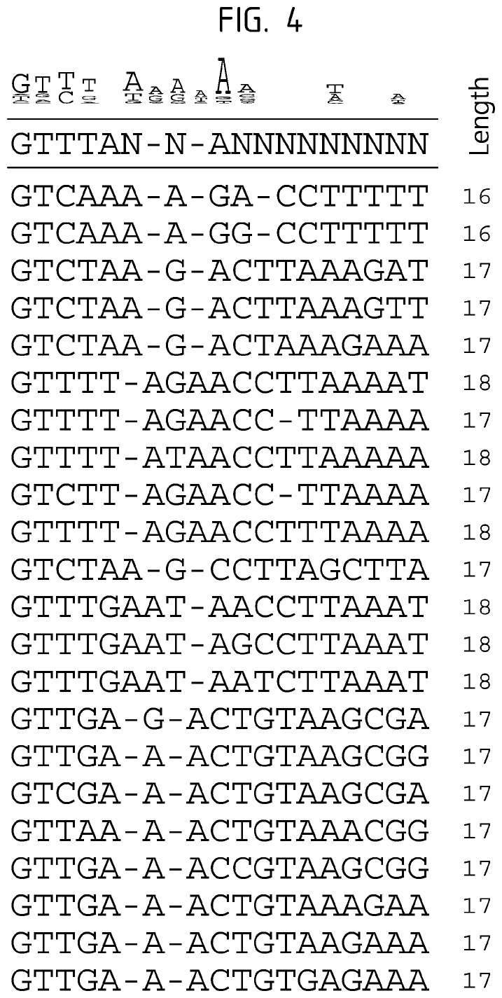

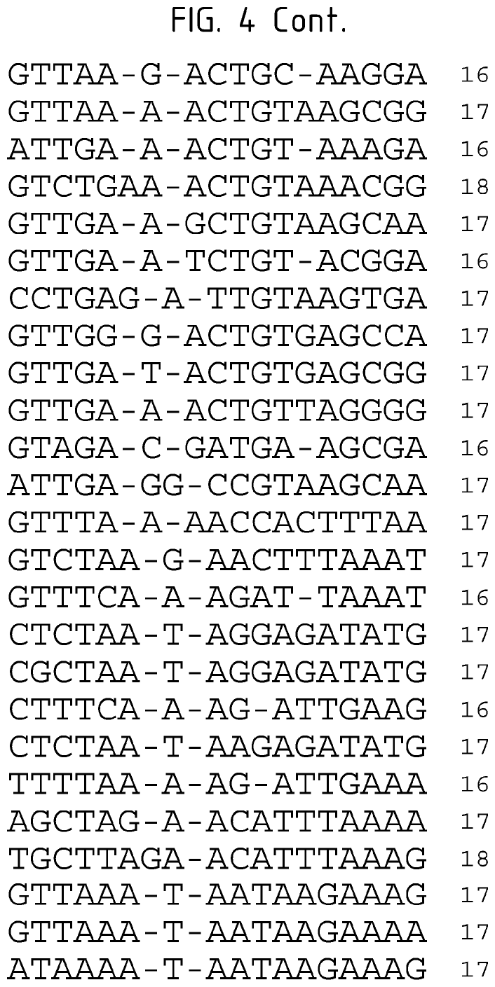

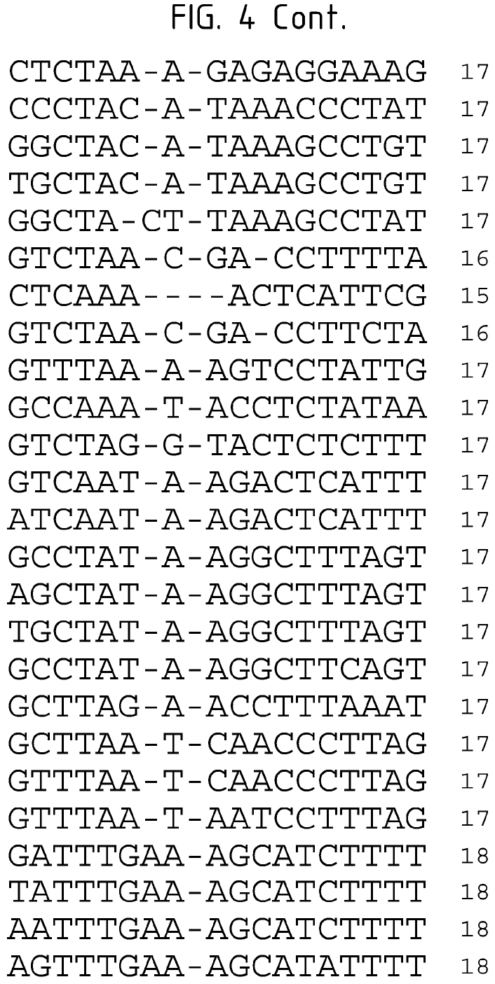

In some embodiments, the separator sequence is about 2 to about 15 nt in length. In some embodiments, the separator sequence is about 3 to about 10 nt in length. In some embodiments, the separator sequence is about 3 to about 9 nt in length. In some embodiments, the separator sequence is about 3 to about 8 nt in length. In some embodiments, the separator sequence is 3, 4, 5, 6, 7, or 8 nt in length. Some non-limiting examples of the separator sequences include AAAT (SEQ ID NO: 3), TTATA (SEQ ID NO: 4), ATTAA (SEQ ID NO: 5), TATAATT (SEQ ID NO: 6), TTTT (SEQ ID NO: 114), TTTA (SEQ ID NO: 115), and ATTT (SEQ ID NO: 116) (FIG. 3H). However, it is noted that a skilled person in the art can optimize the length and the sequence based on use.

In some embodiments, the engineered multiplex CRISPR array is capable of binding to one or more target nucleic acids. As used herein, a “target nucleic acid sequence” of a CRISPR array refers to a sequence to which a spacer sequence is designed to have complementarity, where hybridization between a target nucleic acid sequence and a spacer sequence promotes the formation of a CRISPR complex.

The terms “nucleic acid” and “polynucleotide” are used interchangeably herein, and refer to both ribonucleic acids (RNA) and deoxyribonucleic acids (DNA) molecules, including nucleic acids comprising cDNA, genomic DNA, and/or synthetic DNA, and DNA or RNA molecules containing nucleic acid analogs. A nucleic acid can be double-stranded or single-stranded (for example, a sense strand or an antisense strand). Nucleic acids comprise the nucleotide bases adenine (A), guanine (G), thymine (T), cytosine (C). Uracil (U) replaces thymine in RNA molecules. The symbol “N” can be used to represent any nucleotide base (e.g., A, G, C, T, or U). A nucleic acid may contain unconventional or modified nucleotides. The terms “polynucleotide sequence” and “nucleic acid sequence” as used herein interchangeably refer to the sequence of a nucleic acid molecule. The nomenclature for nucleotide bases set forth in 37 CFR §1.822 is used herein.

In some embodiments, the target nucleic acid refers to a nucleic acid of interest. For instance, in some embodiments, the target nucleic acid refers to a nucleic acid being investigated. In some embodiments, the target nucleic acid is an endogenous gene. In specific embodiments, the target nucleic acids comprise double-stranded DNAs (dsDNAs). In other embodiments, the target nucleic acid is an RNA molecule. In some embodiments, the target nucleic acids comprise RNAs and DNAs.

In some embodiments, the target nucleic acid refers to a genomic site or DNA locus capable of being recognized by and bound to a crRNA provided herein. An enzymatically active crRNA-Cas complex would process such a target site to result in a break at the CRISPR target site. In the case of a deactivated Cas, a crRNA-dCas still recognizes and binds a CRISPR target site without cutting the target nucleic acid (e.g., DNA or RNA).

In some embodiments, the target nucleic acid is a regulatory DNA element, such as but not limited to, a promoter or an enhancer. In some embodiments, the target nucleic acid is part of a gene sequence that can be transcribed into RNA. In some embodiments, the target nucleic acid is part of a transcribed gene sequence that can be translated into protein. In some embodiments, the target nucleic acid comprises a transcription factor. In some embodiments, the target nucleic acid is involved in a pathological pathway, such as but not limited to, cancer or an immune disease. In some embodiments, the target nucleic acid is involved in a biological pathway, such as but not limited to, cell signaling, cell metabolism, aging, cell death, angiogenesis, DNA repair, and stem cell differentiation.

In some embodiments, the engineered multiplex CRISPR array are configured to target a plurality of target nucleic acids simultaneously and the plurality of target nucleic acids comprise RNAs. In some embodiments, the engineered multiplex CRISPR array are configured to target a plurality of target nucleic acids simultaneously and the plurality of target nucleic acids comprise DNAs. In some embodiments, the engineered multiplex CRISPR array are configured to target a plurality of target nucleic acids simultaneously and the plurality of target nucleic acids comprise RNAs and DNAs.

In some embodiments, the engineered multiplex CRISPR array is capable of upregulating the expression of a plurality of target nucleic acids simultaneously. In other embodiments, the engineered multiplex CRISPR array is capable of downregulating the expression of the plurality of target nucleic acids simultaneously. In some embodiments, the engineered multiplex CRISPR array is capable of upregulating and downregulating the expression of the plurality of target nucleic acids simultaneously.

In some exemplary embodiments, an engineered multiplex CRISPR array provided herein comprises a plurality of crRNAs with Cas12a repeat sequences and is capable of upregulating the expression of a plurality of target nucleic acids (e.g., target dsDNAs) simultaneously.

In other exemplary embodiments, an engineered multiplex CRISPR array provided herein comprises a plurality of crRNAs with Cas13 (e.g., Cas13d or Cas13b) repeat sequences and is capable of downregulating the expression of a plurality of target nucleic acids (e.g., target RNAs) simultaneously.

In yet other exemplary embodiments, an engineered multiplex CRISPR array provided herein comprises a plurality of crRNAs with Cas12a repeat sequences and a plurality of crRNAs with Cas13 (e.g., Cas13d or Cas13b) repeat sequences, and is capable of upregulating the expression of a plurality of target nucleic acids (e.g., target dsDNAs) and downregulating the expression of a plurality of target nucleic acids (e.g., target RNAs) simultaneously. In certain embodiments, the plurality of crRNAs with Cas12a repeat sequences, Cas13 (e.g., Cas13d or Cas13b) repeat sequences, or both, are comprised in a single construct.

In some embodiments, the CRISPR array provided herein can target any number of nucleic acids. In some embodiments, the CRISPR array provided herein can target at least 4 different target nucleic acids. In some embodiments, the CRISPR array provided herein can target at least 10 different target nucleic acids. In some embodiments, the CRISPR array provided herein can target at least 15, at least 20, at least 25, at least 30 different target nucleic acids. In some embodiments, the CRISPR array provided herein can target at least 50 different target nucleic acids. In other embodiments, the CRISPR array provided herein can target at least 100 different target nucleic acids.

In some embodiments, the engineered multiplex CRISPR array provided herein is a Cas12a array. In some embodiments, the Cas12a array comprises a plurality of crRNAs in tandem. In some embodiments, each of the crRNAs in the Cas12a array comprises a Cas12a repeat sequence and a spacer, in which each repeat sequence is a Cas12a repeat sequence and each spacer is configured to hybridize to a different target nucleic acid. In some embodiments, each of the Cas12a repeat sequence is immediately preceded by a separator described herein.

In other embodiments, the engineered multiplex CRISPR array provided herein is a Cas13 array. In these embodiments, each of the crRNAs in the Cas13 array comprises a Cas13 repeat sequence (e.g., a Cas13b or Cas13d repeat sequence) and a spacer, in which each repeat sequence is a Cas13 repeat sequence and each spacer is configured to hybridize to a different target nucleic acid. In some embodiments, each of the Cas13 repeat sequence is immediately preceded by a separator described herein.

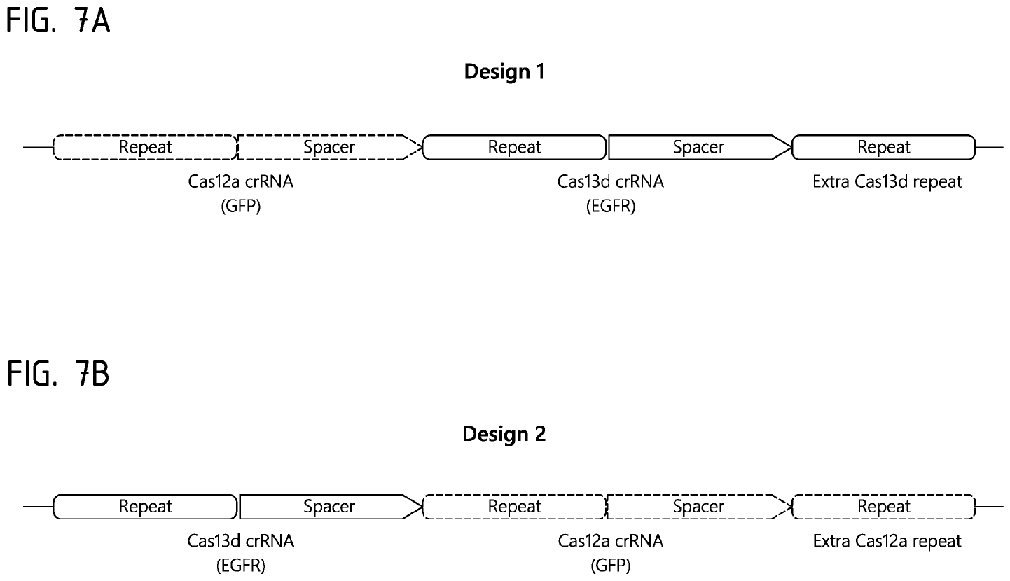

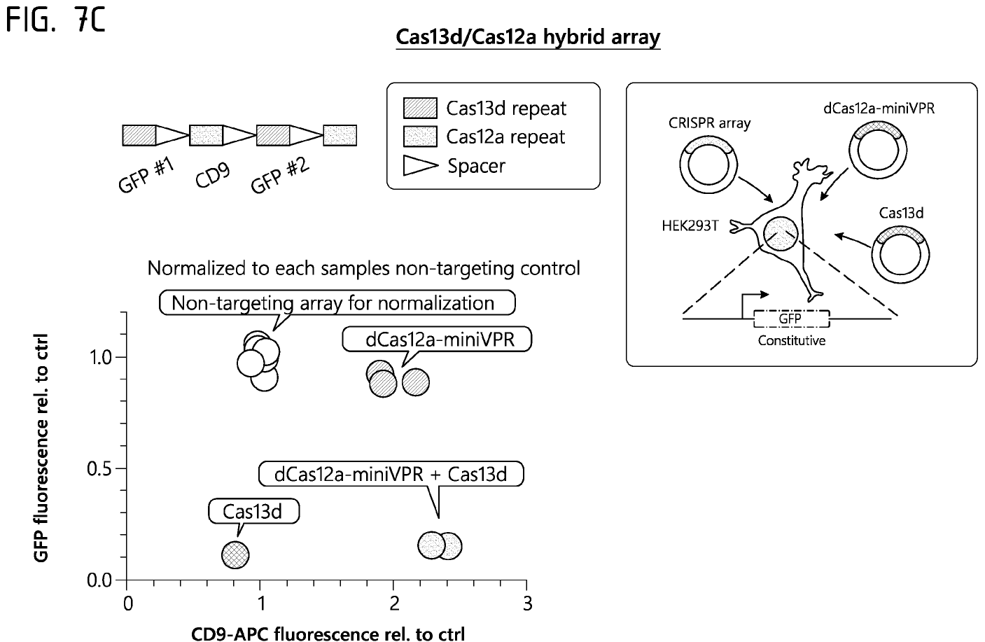

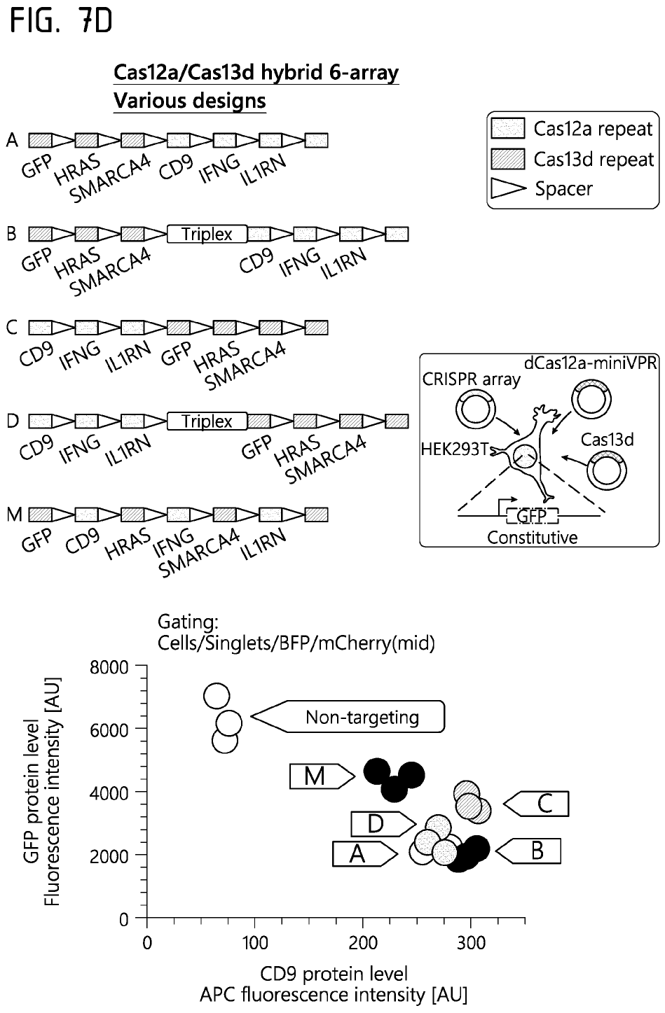

In some embodiments, the engineered multiplex CRISPR array provided herein is a hybrid Cas12a and Cas13 array. In some embodiments, the hybrid Cas12a and Cas13 array comprises one or more Cas12a crRNAs and one or more Cas13 crRNAs as described herein. In certain embodiments, the one or more Cas12a crRNAs precede the one or more Cas13 crRNAs, i.e., all of the one or more Cas12a crRNAs are 5′—to all of the one or more Cas13 crRNAs. A non-limiting exemplary illustration is provided in FIG. 7A. In other embodiments, the one or more Cas13 crRNAs precede the one or more Cas12a crRNAs, i.e., all of the one or more Cas13 crRNAs are 5′—to all of the one or more Cas12a crRNAs. A non-limiting exemplary illustration is provided in FIG. 7B. In other embodiments, the one or more Cas12a crRNAs and Cas13 crRNAs are intermingled with no particular internal order.

### Nucleic Acids and Vectors

An aspect of the disclosure is one or more nucleic acids that encode the engineered multiplex CRISPR array as described herein. As used herein, “encoding” refers to a polynucleotide encoding for the amino acids of a polypeptide or a non-coding RNA molecule. A series of three nucleotide bases encodes one amino acid. As used herein, “expressed,” “expression,” or “expressing” refers to transcription of RNA from a DNA molecule. In some embodiments, the nucleic acid is operably linked to a heterologous nucleic acid sequence, such as, for example a structural gene that encodes a protein of interest or a regulatory sequence (e.g., a promoter sequence). As used herein, the term “operably linked” refers to a functional linkage between a promoter or other regulatory element and an associated transcribable DNA sequence or coding sequence of a gene (or transgene), such that the promoter, etc., operates to initiate, assist, affect, cause, and/or promote the transcription and expression of the associated transcribable DNA sequence or coding sequence, at least in certain tissue(s), developmental stage(s) and/or condition(s). In addition to promoters, regulatory elements include, without being limiting, an enhancer, a leader, a transcription start site (TSS), a linker, 5′ and 3′ untranslated regions (UTRs), an intron, a polyadenylation signal, and a termination region or sequence, etc., that are suitable, necessary or preferred for regulating or allowing expression of the gene or transcribable DNA sequence in a cell. Such additional regulatory element(s) can be optional and used to enhance or optimize expression of the gene or transcribable DNA sequence.

Also provided herein are vectors and/or plasmids containing one or more of the nucleic acids encoding the engineered multiplex CRISPR array as described herein. As used herein, the terms “vector” or “plasmid” are used interchangeably and refer to a circular, double-stranded DNA molecule that is physically separate from chromosomal DNA. In one embodiment, a plasmid or vector used herein is capable of replication in vivo. In one embodiment, a plasmid provided herein is a bacterial plasmid. In one aspect, a plasmid or vector provided herein is a recombinant vector. As used herein, the term “recombinant vector” refers to a vector formed by laboratory methods of genetic recombination, such as molecular cloning. In another embodiment, a plasmid provided herein is a synthetic plasmid. As used herein, a “synthetic plasmid” is an artificially created plasmid that is capable of the same functions (e.g., replication) as a natural plasmid. Without being limited, one skilled in the art can create a synthetic plasmid de novo via synthesizing a plasmid by individual nucleotides, or by splicing together nucleic acids from different pre-existing plasmids. In other embodiments, the vector comprises a viral vector. In some embodiments, the viral vector comprises a lentiviral vector, an adeno virus vector, an adeno-associated viral vector, a piggyBac vector, herpes virus, simian virus 40 (SV40), bovine papilloma virus vectors, or a retroviral vector. Some embodiments disclosed herein relate expression cassettes including a nucleic acid molecule as disclosed herein.

In other embodiments, the present disclosure also provides expression cassettes containing one or more of the nucleic acids encoding the engineered multiplex CRISPR array as described herein. An expression cassette is a construct of genetic material that contains coding sequences and enough regulatory information to direct proper transcription and/or translation of the coding sequences in a recipient cell, in vivo and/or ex vivo. The expression cassette may be inserted into a vector for targeting to a desired host cell. As such, the term “expression cassette” may be used interchangeably with the term “expression construct.”

A host cell as used herein can be a eukaryotic cell or prokaryotic cell. Non-limiting examples of eukaryotic cells include animal cell, plant cells, and fungal cells. In some embodiment, the eukaryotic cell comprises CHO, HEK293T, Sp2/0, MEL, COS, and insect cells. In some embodiment, the eukaryotic cell comprises mammalian cells. In some embodiment, the eukaryotic cell comprises human cells. In some embodiment, the prokaryotic cells include, but are not limited to, E. coli.

In some embodiments, the vector provided herein further comprises a promoter. As used herein, the term “promoter” generally refers to a DNA sequence that contains an RNA polymerase binding site, transcription start site, and/or TATA box and assists or promotes the transcription and expression of an associated transcribable polynucleotide sequence and/or gene (or transgene). A promoter can be synthetically produced, varied or derived from a known or naturally occurring promoter sequence or other promoter sequence. A promoter can also include a chimeric promoter comprising a combination of two or more heterologous sequences. A promoter of the present application can thus include variants of promoter sequences that are similar in composition, but not identical to, other promoter sequence(s) known or provided herein. A promoter can be classified according to a variety of criteria relating to the pattern of expression of an associated coding or transcribable sequence or gene (including a transgene) operably linked to the promoter, such as constitutive, developmental, tissue-specific, inducible, etc. Promoters that drive expression in all or most tissues of the plant are referred to as “constitutive” promoters. Promoters that drive expression during certain periods or stages of development are referred to as “developmental” promoters. Promoters that drive enhanced expression in certain tissues of the plant relative to other plant tissues are referred to as “tissue-enhanced” or “tissue-preferred” promoters. Thus, a “tissue-preferred” promoter causes relatively higher or preferential expression in a specific tissue(s) of the plant, but with lower levels of expression in other tissue(s) of the plant. Promoters that express within a specific tissue(s) of the plant, with little or no expression in other plant tissues, are referred to as “tissue-specific” promoters. An “inducible” promoter is a promoter that initiates transcription in response to an environmental stimulus such as cold, drought or light, or other stimuli, such as wounding or chemical application. A promoter can also be classified in terms of its origin, such as being heterologous, homologous, chimeric, synthetic, etc. A “heterologous” promoter is a promoter sequence having a different origin relative to its associated transcribable sequence, coding sequence, or gene (or transgene), and/or not naturally occurring in the plant species to be transformed. In some embodiments, the promoter comprises a polymerase II promoter. In some embodiments, the polymerase II promoter comprises a CAG promoter avPGK promoter, an EF1a promoter, and a SFFV promoter.

In some embodiments, the vector provided herein further comprises a reporter gene. In some embodiments, the reporter gene comprises BFP, GFP, and mCherry.

The nucleic acids described herein can be contained within a vector that is capable of directing their expression in, for example, a cell that has been transduced with the vector. Suitable vectors for use in eukaryotic cells are known in the art and are commercially available or readily prepared by a skilled artisan. Additional vectors can also be found, for example, in Ausubel, F. M., et al., Current Protocols in Molecular Biology, (Current Protocol, 1994) and Sambrook et al., “Molecular Cloning: A Laboratory Manual,” 2nd Ed. (1989).

The vectors are useful for autonomous replication in a host cell or may be integrated into the genome of a host cell upon introduction into the host cell, and thereby are replicated along with the host genome (e.g., non-episomal mammalian vectors).

In some embodiments, the vector is an expression vector. Expression vectors are capable of directing the expression of coding sequences to which they are operably linked. In some embodiments, the vector is eukaryotic expression vector, i.e. the vector is capable of directing the expression of coding sequences to which they are operably linked in a eukaryotic cell. In general, expression vectors of utility in recombinant DNA techniques are often in the form of plasmids (vectors). However, other forms of expression vectors, such as viral vectors (e.g., replication defective retroviruses, adenoviruses, and adeno-associated viruses) are also included.

DNA vectors can be introduced into eukaryotic cells via conventional transformation or transfection techniques. Suitable methods for transforming or transfecting host cells can be found in Sambrook et al. (1989) Molecular Cloning: A Laboratory Manual (2nd ed., Cold Spring Harbor Laboratory Press, Plainview, N.Y.) and other standard molecular biology laboratory manuals.

In some embodiments, the vector is a viral vector. The term “viral vector” is widely used to refer either to a nucleic acid molecule that includes virus-derived nucleic acid elements that typically facilitate transfer of the nucleic acid molecule or integration into the genome of a cell, or to a viral particle that mediates nucleic acid transfer. Viral particles typically include viral components, and sometimes also host cell components, in addition to nucleic acid(s). Retroviral vectors used herein contain structural and functional genetic elements, or portions thereof, that are primarily derived from a retrovirus. Retroviral lentivirus vectors contain structural and functional genetic elements, or portions thereof including LTRs, that are primarily derived from a lentivirus (a sub-type of retrovirus).

In some embodiments, the nucleic acids are delivered by non-viral delivery vehicles known in the art. For example, the nucleic acid molecule can be stably integrated in the host genome, or can be episomally replicating, or present in the recombinant host cell as a mini-circle expression vector for stable or transient expression. Accordingly, in some embodiments disclosed herein, the nucleic acid molecule is maintained and replicated in the recombinant host cell as an episomal unit. In some embodiments, the nucleic acid molecule is stably integrated into the genome of the recombinant cell. Stable integration can also be accomplished using classical random genomic recombination techniques or with more precise genome editing techniques such as using guide RNA-directed CRISPR/Cas9, DNA-guided endonuclease genome editing NgAgo (Natronobacterium gregoryi Argonaute), or TALENs genome editing (transcription activator-like effector nucleases). In some embodiments, the nucleic acid molecule is present in the recombinant host cell as a mini-circle expression vector for stable or transient expression.

The nucleic acids can be encapsulated in a viral capsid or a lipid nanoparticle. For example, introduction of nucleic acids into cells may be achieved using viral transduction methods. In a non-limiting example, adeno-associated virus (AAV) is a non-enveloped virus that can be engineered to deliver nucleic acids to target cells via viral transduction. Several AAV serotypes have been described, and all of the known serotypes can infect cells from multiple diverse tissue types. AAV is capable of transducing a wide range of species and tissues in vivo with no evidence of toxicity, and it generates relatively mild innate and adaptive immune responses.

Lentiviral systems are also useful for nucleic acid delivery and gene therapy via viral transduction. Lentiviral vectors offer several attractive properties as gene-delivery vehicles, including: (i) sustained gene delivery through stable vector integration into the host cell genome; (ii) the ability to infect both dividing and non-dividing cells; (iii) broad tissue tropisms, including important gene- and cell-therapy-target cell types; (iv) no expression of viral proteins after vector transduction; (v) the ability to deliver complex genetic elements, such as polycistronic or intron-containing sequences; (vi) a potentially safer integration site profile (e.g., by targeting a site for integration that has little or no oncogenic potential); and (vii) a relatively easy system for vector manipulation and production.

### Engineered Cells

Another aspect of the present disclosure encompasses engineered cells. In some embodiments, the engineered multiplex CRISPR arrays described herein are used in eukaryotic cells, such as mammalian cells, for example, human cells, to produce engineered cells with modulated expression of target nucleic acids. Any human cell is contemplated for use with the engineered multiplex CRISPR arrays disclosed herein.

In some embodiments, the cells are engineered to express one or more Cas nucleases. In some embodiments, the engineered cells express Cas12 proteins. In some embodiments, the engineered cells express Cas13 proteins (e.g., Cas13b and/or Cas13d proteins). In other embodiments, the engineered cells express Cas12 and Cas13 (e.g., Cas13b and/or Cas13d) proteins.

In some embodiments, an engineered cell ex vivo or in vitro includes: (a) nucleic acid encoding engineered multiplex CRISPR arrays; and/or (b) one or more Cas nucleases described herein.

Some embodiments disclosed herein relate to a method of engineering a cell that includes introducing into the cell, such as an animal cell, the engineered multiplex CRISPR arrays as described herein, and selecting or screening for an engineered cell transformed by the engineered multiplex CRISPR arrays. The term “engineered cell” refers not only to the particular subject cell but also to the progeny or potential progeny of such a cell. Because certain modifications may occur in succeeding generations due to either mutation or environmental influences, such progeny may not, in fact, be identical to the parent cell, but are still included within the scope of the term as used herein. Techniques for transforming a wide variety of cell are known in the art.

In a related aspect, some embodiments relate to engineered cells, for example, engineered animal cells that include a heterologous nucleic acid and/or polypeptide as described herein. The nucleic acid can be stably integrated in the host genome, or can be episomally replicating, or present in the engineered cell as a mini-circle expression vector for stable or transient expression.

In some embodiments, provided herein is an engineered cell, e.g., an isolated engineered cell, prepared by modulating the expression of a target gene in a target nucleic acid or otherwise modifying the target nucleic acid in a cell according to any of the methods described herein, thereby producing the engineered cell. In some embodiments, provided herein is an engineered cell prepared by a method comprising providing to a cell an engineered multiplex CRISPR array as described herein.

In some embodiments, according to any of the engineered cells described herein, the engineered cell is capable of expressing or not expressing target nucleic acids (e.g., target genes). In some embodiments, according to any of the engineered cells described herein, the engineered cell is capable of regulated expression of target nucleic acids (e.g., target genes). In some embodiments, according to any of the engineered cells described herein, the engineered cell exhibits altered expression pattern of target nucleic acids (e.g., target genes). In other embodiments, the engineered cells described herein exhibits desired phenotypes because of the altered expression pattern of target nucleic acids (e.g., target genes).

### Kits

In some embodiments, provided herein are kits for carrying out a method described herein. A kit can include one or more components of the engineered multiplex CRISPR array as described herein. In some embodiments, the engineered multiplex CRISPR array comprises more than one crRNAs, wherein each of the more than one crRNAs comprises a repeat sequence and a spacer, wherein the spacer is configured to hybridize to a specific target nucleic acid of a plurality of target nucleic acids, and wherein the repeat sequence in each of the more than one crRNAs is preceded by a separator sequence.

A kit as described herein can further include one or more additional reagents, where such additional reagents can be selected from: a buffer for introducing one or more components of an engineered multiplex CRISPR array into a cell; a dilution buffer; a reconstitution solution; a wash buffer; a control reagent; a control expression vector or polyribonucleotide; a reagent for in vitro production of one or more components of an engineered multiplex CRISPR array, and the like.

Components of a kit can be in separate containers; or can be combined in a single container.

In addition to the above-mentioned components, a kit can further include instructions for using the components of the kit to practice the methods. The instructions for practicing the methods are generally recorded on a suitable recording medium. For example, the instructions may be printed on a substrate, such as paper or plastic, etc. As such, the instructions may be present in the kits as a package insert, in the labeling of the container of the kit or components thereof (e.g., associated with the packaging or sub-packaging) etc. In some embodiments, the instructions are present as an electronic storage data file present on a suitable computer readable storage medium, e.g., CD-ROM, diskette, flash drive, etc. In yet other embodiments, the actual instructions are not present in the kit, but means for obtaining the instructions from a remote source, e.g., via the internet, are provided. An example of this embodiment is a kit that includes a web address where the instructions can be viewed and/or from which the instructions can be downloaded. As with the instructions, this means for obtaining the instructions is recorded on a suitable substrate.

### III. Methods of Making Engineered Multiplex CRISPR Arrays

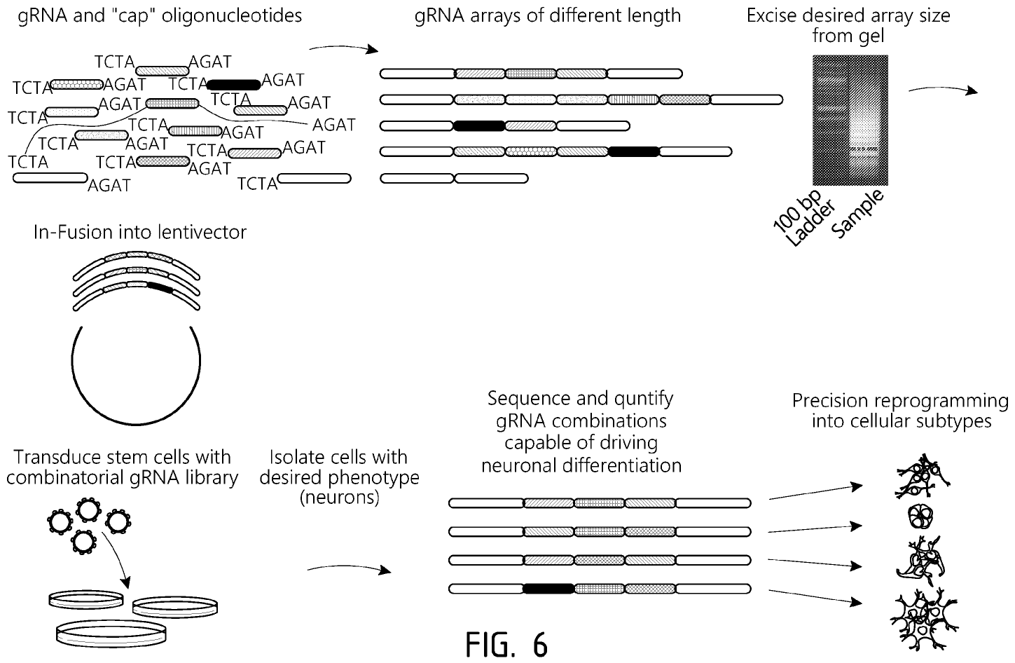

Another aspect of the present disclosure encompasses a method of making a collection of engineered multiplex CRISPR arrays. An exemplary, non-limiting illustration of the major steps of method is provide in FIG. 6. In some embodiments, the method comprises providing more than one crRNAs, wherein each of the more than one crRNAs comprises a 5′ oligonucleotide overhang and a 3′ oligonucleotide overhang configured to hybridize to each other. In some embodiments, each of the more than one crRNAs comprises a repeat sequence and a spacer, wherein the spacer is configured to hybridize to a specific target nucleic acid of a plurality of target nucleic acids, and wherein the repeat sequence in each of the more than one crRNAs is preceded by a separator sequence. In other embodiments, the method further comprises randomly hybridizing the more than one crRNAs to generate the collection of the engineered multiplex CRISPR arrays. An Example procedure for assembling the CRISPR arrays of the present disclosure is provided in Example 2.

In some embodiments, the method further comprises identifying the collection of engineered multiplex CRISPR arrays having a desired length. Methods for identifying the desired nucleic acids are commonly known in the art. For example, the length of nucleic acid fragment can be determined by agarose gel electrophoresis. In some embodiments, the fragments with the desired length are excised and the nucleic acid (e.g., DNA) samples recovered from the agarose gel, resulting in a collection of the desired engineered multiplex CRISPR arrays. In some embodiments, the method further comprises inserting each of the collection of the engineered multiplex CRISPR arrays into a vector.

However, other equivalent methods are known in the art and can be used to achieve the same purpose, and therefore are also encompassed by the present disclosure.

In other embodiments, the method further comprises delivering the collection of the engineered multiplex CRISPR arrays into host cells. In some embodiments, the host cells express one or more Cas proteins. For example, in some embodiments, the host cell express Cas12a proteins. In other embodiments, the host cell express Cas13 proteins. In some embodiments, the host cell express Cas13b proteins. In some embodiments, the host cell express Cas13d proteins. In some embodiments, the host cell express both Cas12a and Cas13 (e.g., Cas13b and/or Cas13d) proteins.

In some embodiments, the method further comprises screening for the collection of engineered multiplex CRISPR arrays with a desired phenotype. Non-limiting exemplary desired phenotypes include immune-evasion in natural killer (NK) cells, simultaneous upregulation (e.g., activation) of the expression of multiple target nucleic acids, simultaneous downregulation (e.g., silencing) of the expression of multiple target nucleic acids, or simultaneous upregulation and downregulation (e.g., simultaneous activation and silencing) of the expression of multiple target nucleic acids, stem cell differentiation patterns, enhanced tumor/cancer killing, modified cell signaling properties, and modified metabolic properties. In certain embodiments, the desired phenotype can be controlled stem cell differentiation, controlled killing of tumor cells, and enhanced cell proliferation, increased T-cell activity level, modified metabolic activity, modified drug sensitivity, modified cell reprogramming efficacy, modified structure and behavior of organelles or cellular subcompartments, modified transcription, and/or translation properties.

In other embodiments, the screening further comprises isolating the host cells exhibiting the desired phenotype. In some embodiments, the method further comprises sequencing the engineered multiplex CRISPR array expressed by the isolated host cells. In some embodiments, the method further comprises isolating the desired engineered multiplex CRISPR array. In other embodiments, the isolated desired engineered multiplex CRISPR arrays can be used in various applications or methods, such as but not limited to those described herein.

### IV. Methods of Targeting Nucleic Acids

Provided herein are methods of targeting (e.g., binding to, modifying, detecting, etc.) one or more target nucleic acids (e.g., dsDNA or RNA) using the engineered multiplex CRISPR array provided herein.

In some embodiments, provided herein is a method of targeting (e.g., binding to, modifying, detecting, etc.) a target nucleic acid in a sample comprising introducing into the sample the components of the engineered multiplex CRISPR array as described herein. A sample as used here can be a biological sample comprising a cell, including, without limitation, a tissue, fluid, or other composition in an organism. In some embodiments, the sample is a cell or a composition comprising a cell. In some embodiments, the cell is a mammalian cell, e.g., a human cell.

Targeting a nucleic acid molecule can include one or more of cutting or nicking the target nucleic acid molecule; modulating the expression of a gene present in the target nucleic acid molecule (such as by regulating transcription of the gene from a target DNA or RNA, e.g., to downregulate and/or upregulate expression of a gene); visualizing, labeling, or detecting the target nucleic acid molecule; binding the target nucleic acid molecule, editing the target nucleic acid molecule, trafficking the target nucleic acid molecule, and masking the target nucleic acid molecule. In some embodiments, modifying the target nucleic acid molecule includes introducing one or more of a nucleobase substitution, a nucleobase deletion, a nucleobase insertion, a break in the target nucleic acid molecule, methylation of the target nucleic acid molecule, and demethylation of the nucleic acid molecule. In some embodiments, such methods are used to treat a disease, such as a disease in a human. In such embodiments, one or more target nucleic acids are associated with the disease.

### V. Methods of Gene Modulation

In some embodiments, the engineered multiplex CRISPR array provided herein can be used to control endogenous gene expression. In some embodiments, the present disclosure describes a method for improving multi-gene control in host cells, e.g., human cells. In some embodiments, the present disclosure provides a crucial component of the molecular toolkit that enables high-precision control of cell identity, cell differentiation pattern, and/or cell behavior.

In some embodiments, the present disclosure provides a method for controlled stem cell differentiation comprising contacting a stem cell with a plurality of the engineered multiplex CRISPR arrays comprising crRNAs configure to hybridize to target genes known to influence the stem cell identity.

In other embodiments, the present disclosure provides a method for simultaneous activation of multiple endogenous genes. In some embodiments, the method comprises contacting a host cell with the engineered multiplex CRISPR array provided herein. In certain embodiments, the more than one crRNAs in the CRISPR array comprise Cas12a repeat sequences and spacers configured to hybridize to a plurality of target nucleic acids. One exemplary embodiment is shown in FIG. 3D and Example 5.

In some embodiments, the present disclosure provides a method for simultaneous silencing of multiple endogenous genes. In some embodiments, the method comprises contacting a host cell with the engineered multiplex CRISPR array provided herein, in which the more than one crRNAs comprise Cas13 repeat sequences and spacers configured to hybridize to a plurality of target nucleic acids.

In other embodiments, the present disclosure provides a method for simultaneous activation and silencing of multiple endogenous genes. In these embodiments, the method comprises contacting a host cell with the engineered multiplex CRISPR array provided herein, in which the more than one crRNAs comprise both Cas12a and Cas13 repeat sequences and spacers configured to hybridize to a plurality of target nucleic acids.

In some embodiments, the host cells express one or more Cas proteins. For example, in some embodiments, the host cell express Cas12a proteins. In other embodiments, the host cell express Cas13 proteins. In some embodiments, the host cell express Cas13b proteins. In some embodiments, the host cell express Cas13d proteins. In some embodiments, the host cell express both Cas12a and Cas13b proteins.

### EXAMPLES

The practice of the present invention will employ, unless otherwise indicated, conventional techniques of molecular biology, microbiology, cell biology, biochemistry, nucleic acid chemistry, and immunology, which are well known to those skilled in the art.

Additional embodiments are disclosed in further detail in the following examples, which are provided by way of illustration and are not in any way intended to limit the scope of this disclosure or the claims.

### Example 1: Materials and Methods

The purpose of this Example is to provide the exemplary materials and methods that were used herein.

HEK293T cells (Clontech) carrying a genomically integrated dscGFP gene driven by the TRE3G promoter (consisting of seven repeats of the Tet response element) were used. This cell line was clonally sorted and expanded and showed no background GFP fluorescence. Cells were cultured in DMEM+GlutaMAX (Thermo Fisher) containing 100 U/mL of penicillin and streptomycin (Life Technologies) and 10% Fetal Bovine Serum (Clontech). Cells were grown at 37° C. with 5% CO₂ and passaged using 0.05% Trypsin-EDTA solution (Thermo Fisher).

For Example 6, HEK293T cells (Takara Bio, Japan) were engineered to carry a genomically integrated GFP gene driven by the TRE3G promoter (consisting of seven repeats of the Tet response element), and a Tet3G activator driven by the EF1a promoter. Cells were cultured in DMEM+GlutaMAX (Thermo Fisher, Waltham, MA) containing 100 U/ml of penicillin and streptomycin (Thermo Fisher) and 10% fetal bovine serum (Clontech). Cells were grown at 37° C. with 5% CO2 and passaged using 0.05% Trypsin-EDTA solution (Thermo Fisher) or TryplE Express Enzyme (Thermo Fisher).

Cells were transfected with constructs carrying 1) nuclease-deactivated Cas12a (from Lachnospiraceae bacterium, human codon-optimized) fused either to the VP64-p65-Rta (VPR) activator and mCherry, or to mini-VPR and mCherry; 2) a CRISPR array-expressing plasmid. For FIGS. 1 and 3A-C, a CRISPR array construct consisting of firefly luciferase immediately followed by a CRISPR array and an SV40 pA terminator, expressed under the CAG promoter element was used. For the activation of seven endogenous genes (FIG. 3D), firefly luciferase was replaced with Blue Fluorescent Protein (BFP), a Malat1 Triplex sequence, and the L. bacterium Cas12a leader sequence.

Cells were seeded one day before transfection at a density of 5×10⁴ cells per well in a 24-well plate. Cells were transfected using TransIT-LT1 transfection reagent (Minis Bio, Madison, WI) according to the manufacturer's recommendation (250 ng dCas12a-VPR-mCherry plasmid; 250 ng CRISPR array plasmid; 1.5 μl transfection reagent per well).

Two days after transfection, cells were dissociated using 0.05% Trypsin-EDTA (Thermo Fisher), passed through a 40 μm filter-capped test tube (Corning), and analyzed using a CytoFLEX S flow cytometer (Beckman Coulter). For each experiment, 10,000 events were recorded.

For Example 6, On day 0, cells were seeded 1 day before transfection at a density of 4×10⁴ cells per well in a 48-well plate. On day 1, cells were transfected with constructs carrying (1) nuclease-deactivated dCas12a (from L. bacterium, human codon-optimized) fused to the mini-VPR activator (Vora et al., 2018) and mCherry; (2) Cas13d from Ruminococcus flavefaciens (Konermann et al., Cell, 2017) followed by a 2A element and mCherry, driven by the EF1a promoter (3) a CRISPR array-expressing plasmid. On day 2, medium was changed to medium including doxycycline (1 ug/ml) to activate endogenous GFP expression. On day 3, cells were dissociated using TrypLE (Thermo Fisher), centrifuged at 300*g for 5 minutes after which the supernatant was removed. Cells were incubated with an APC-conjugated CD9-targetingand antibody (BD Biosciences) at a 1:100 dilution for 2 hours at 4′C. Cells were then centrifuged at 300*g for 5 minutes after which the supernatant was removed and cells were suspended in PBS and passed through a 40 μm filter-capped test tube (Corning, Corning, NY). Cells were then analyzed using a BD Influx FACS machine (BD Biosciences, Franklin Lakes, NJ). During flow cytometry analysis, cells were gated for expressing the Cas12a construct (mCherry+) and CRISPR array (BFP).

RT-qPCR was conducted to quantify endogenous gene activation. Cells were transfected and harvested as described above. Total RNA was extracted with the RNeasy Plus Mini Kit (QIAGEN), according to manufacturer's instructions. Reverse transcription was performed using iScript cDNA Synthesis kit (Bio-Rad). Quantitative PCR reactions were run on a LightCycler thermal cycler (Bio-Rad) with iTaq Universal SYBR Green Supermix (Bio-Rad). ΔΔCt values for the target genes were divided by those of RPL13A to obtain relative expression. Piimers used in the RT-gPCR were listed in Table 1 below:

TABLE 1
Primers used in the RT-qPCR

| | Sequence |
|---|---|
| Primer name | (SEQ ID NO) |
| has-RPL13A-F | CCTGGAGGAGAAGAGGAAAGAGA |
| | (SEQ ID NO: 7) |
| has-RPL13A-R | TTGAGGACCTCTGTGTATTTGTCAA |
| | (SEQ ID NO: 8) |
| has-CD9-F | CACCAAGTGCATCAAATACCTG |
| | (SEQ ID NO: 9) |
| has-CD9-R | GTTTCTTGCTCGAAGATGCTC |
| | (SEQ ID NO: 10) |
| has-IFNG-F | TCGGTAACTGACTTGAATGTCCA |
| | (SEQ ID NO: 11) |
| has-IFNG-R | TCGCTTCCCTGTTTTAGCTGC |
| | (SEQ ID NO: 12) |
| has-EGFR-F | TTGCCGCAAAGTGTGTAACG |
| | (SEQ ID NO: 13) |
| has-EGFR-R | GTCACCCCTAAATGCCACCG |
| | (SEQ ID NO: 14) |
| has-TMEM107-F | GAGTCTCCATGTTCAACAGCA |
| | (SEQ ID NO: 15) |
| has-TMEM107-R | TCCCAACGCTCGAATATGAAG |
| | (SEQ ID NO: 16) |
| has-IMPACT-F | TACAGCTCCACCTATCTACCA |
| | (SEQ ID NO: 17) |
| has-IMPACT-R | ACATCTCTTATTTTCTCCACCCA |
| | (SEQ ID NO: 18) |
| has-DANCR-F | AGGAGTTCGTCTCTTACGTCT |
| | (SEQ ID NO: 19) |
| has-DANCR-R | TGAAATACCAGCAACAGGACA |
| | (SEQ ID NO: 20) |
| dscGFP-F | ACTTCAAGAGCGCCATCCA |
| | (SEQ ID NO: 21) |
| dscGFP-R | GTCTTGAAGGCGTGCTGGTA |
| | (SEQ ID NO: 22) |

Exemplary spacer sequences used to activate endogenous genes are provided in Table 2 below.

TABLE 2
Exemplary Spacer sequences

| Target | |
|---|---|
| gene | Spacer sequence (SEQ ID NO) |
| CD9 | AAAAGTGCCACTCCTTAGGG (SEQ ID NO: 23) |
| IFNG | AGATGAGATGGTGACAGATA(SEQ ID NO: 24) |
| EGFR | CTCCAGAGCCCGACTCGCCG (SEQ ID NO: 25) |
| TMEM107 | TCGGCTTGCGGGGAGACTTC (SEQ ID NO: 26) |
| IMPACT | CACCCTTCGGCCCGCCACCC (SEQ ID NO: 27) |
| DANCR | AGAAAGGGAATCCCAGGGCC (SEQ ID NO: 28) |
| TRE3G | CTCCCTATCAGTGATAGAGA (SEQ ID NO: 29) |
| (GFP) | |

### Example 2: Assembly of CRISPR Arrays

This Example illustrates how the CRISPR arrays used in the present disclosure were assembled.

CRISPR arrays were assembled using an oligonucleotide duplexing and ligation method. First, arrays were designed computationally using SnapGene. The arrays were designed to include two flanking sequences containing a 20-bp overlap with the opened backbone plasmid, as required for a subsequent In-Fusion reaction. This double-stranded sequence was then inputted into a custom R script that divided the sequence into ≤60-nt single-stranded DNA sequences with unique 4-nt 5′ overhangs, which were ordered from Integrated DNA Technologies (IDT) in LabReady formulation (i.e., 100 μM in IDTE buffer, pH 8.0) and standard desalting purification. For assembly, up to 8 oligo duplexes (i.e. 16 single-stranded oligonucleotides were ligated per reaction vial. For CRISPR arrays longer than that, the first step of the assembly reaction was divided into multiple vials, each ligating ≤8 oligonucleotide duplexes (e.g. if the array consists of 12 oligonucleotide duplexes, perform the reaction in two vials with 6 duplexes in each). For each ligation vial, first make an oligonucleotide mix containing 1 μl of each oligonucleotide. Then set up the following phosphorylation/duplexing reaction:

Phosphorylation and duplexing

| | | |
|---|---|---|
| Oligonucleotide mix | 1.0 | μl |
| 2x T7 ligation buffer | 2.5 | μl |
| H2O | 1.25 | μl |
| T4 PNK | 0.25 | μl |
| Total | 5 | μl |

Then run a phosphorylation-duplexing reaction on a thermocycler using the program below:

| | |
|---|---|
| 37° C. | 30 min |
| 95° C. | 5 min |
| 25° C. | step down 0.1° C./second |
| 25° C. | hold |

Then, add 1 reaction volume (5 μl) of 1× T7 buffer. Add 1 μl T7 DNA ligase (New England Biolabs, MA, USA) (Important: Use T7 ligase rather than T4 ligase, as T7 ligase lacks the ability to ligate blunt ends). Incubate at 25° C. for 3 hours. Then, dilute the sample ⅕ by adding 40 μl water. Run the sample on a 2% agarose gel. A ladder pattern should be visible. Excise the band corresponding to the ligated product. Depending on whether the entire CRISPR array was assembled in a single vial, or divided into several vials, do either of the following:

If the entire array was assembled in a single vial: Gel-purify the excised band using the Macherey-Nagel NucleoSpin Gel & PCR Clean-up kit (Macherey-Nagel, Germany). Insert the purified array into the opened backbone using In-Fusion cloning (Takara Bio, Japan).

If the array was divided into >1 vial: For all excised bands belonging to the same array, pool the excised bands into a single vial. Gel-purify the pooled bands using the Macherey-Nagel NucleoSpin Gel & PCR cleanup kit. Elute in 15 μl water. Then, add 1 volume (15 μl) of 2× T7 buffer and 1 μl T7 DNA ligase. Incubate at 25° C. for 3 hours. Then, run the ligated product on a 2% agarose gel. A faint band should be seen corresponding to the full-length CRISPR array. Excise and gel-purify this band. Insert into backbone vector using In-Fusion.

### Quantification and Statistical Analyses

### Computation of GC Content in Sliding Window

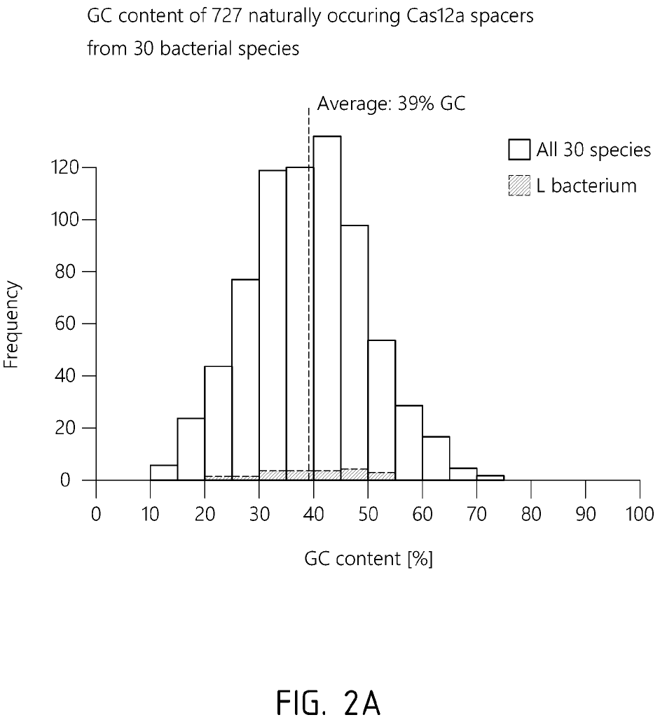

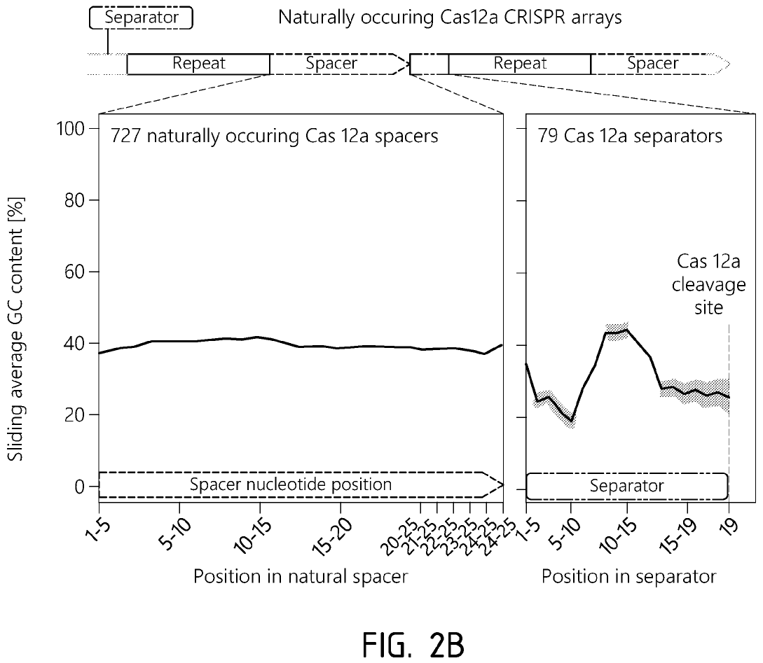

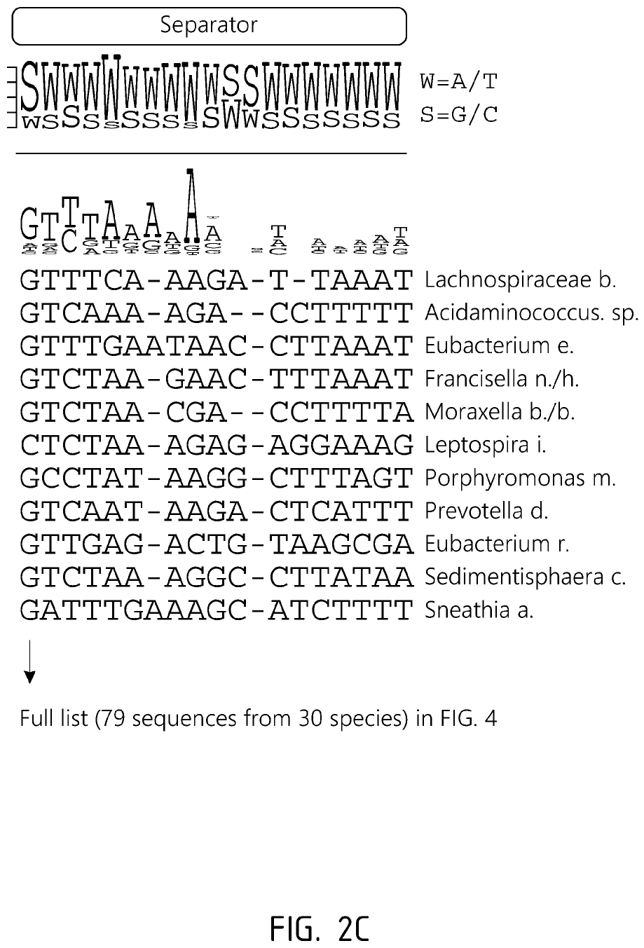

For each of the spacer sequences, the GC content was computed in a sliding 5-nt window (e.g., first nucleotides 1-5, then nucleotides 2-6, etc.). For each of such window, the average and standard error of all 51 spacers were calculated. As the sliding window approached the 3′ end of the spacers, the size of the sliding window was reduced to 4, then 3, then 2 nucleotides, in order to increase resolution at the very 3′ end. This was also performed for naturally occurring spacers and CRISPR separators (FIG. 2B). The analyzed spacers varied in length from 25-36 nt. For this analysis, the 5′ ends of spacers longer than 25 nt were truncated so that the 25 nucleotides at the most 3′ end of every spacer could be aligned and analyzed.

The separator sequences were first aligned using the T-Coffee alignment tool (SnapGene v. 5.2.), which did not truncate any of the separator sequences. For calculating the predictive power of knowing the GC content of 3 bases in the spacer (FIG. 1J), each 20-nt spacer was divided into 18 3-nt windows and the GC content was calculated for each window. For each such window (e.g. window 1-3), the GC content was plotted versus the GFP activation of all 51 arrays (percentage of GFP+cells). Next, a linear regression was performed and the R² value was inserted into FIG. 1J.

### Multiple Sequence Alignment of Naturally Occurring CRISPR Sequences

The multiple sequence alignment tools SnapGene (v. 5.1-5.2) were used for the alignment of separator sequences and post-processed repeats. The separator sequences were aligned using T-Coffee The other sequences were aligned using MUltiple Sequence Comparison by Log-Expectation (MUSCLE).

### Example 3: The GC Content of Spacers Affects Performance of the Downstream crRNA in Cas12a CRISPR Arrays

The purpose of this example is to demonstrate the GC content of spacers affects performance of the downstream crRNA in Cas12a CRISPR arrays.

Short CRISPR arrays with 2 crRNAs were designed to test the effect of GC content of upstream spacer. The 51 spacer sequences (FIG. 1G) were adapted from a negative-control sgRNA library generated by Gilbert et al. (Cell, 2014). These sequences correspond to scrambled Cas9 spacer sequences, and were adjusted slightly for length (20 nt) and GC content.

It was hypothesized that the separator sequence is important for proper processing of the CRISPR array. Because new spacers are excised from viral sequences, it is possible that some spacers will by chance generate RNA secondary structures that sterically hinder Cas12a from accessing its cleavage site. RNA secondary structure is known to impede Cas protein binding and processing. For example, it was known that the RNA-binding and -cleaving protein Cas13 is negatively affected by secondary structure. (Abudayyeh et al., Science, 2016; Yan et al., Mol Cell, 2018). Further, Cas12a is sensitive to a hairpin structure that forms immediately downstream of the CRISPR array (Liao et al., RNA Biology, 2019). It is therefore plausible that local secondary structure within the transcribed CRISPR array itself could interfere with proper array processing (FIG. 1C).

One feature that promotes RNA secondary structure formation is high GC content (Chan et al., BMC Bioinformatics, 2009). Thus, a simple Cas12a array was designed to consist of two consecutive crRNAs whose repeat regions did not contain the separator sequence (FIG. 1D). In this array, the spacer of the second crRNA was complementary to the promoter region of GFP, which had been genomically integrated into HEK293T cells (FIG. 1E). The first crRNA's spacer instead consisted of a non-targeting sequence. Surprisingly, it was discovered that this array design displayed hypersensitivity to the last nucleotide in the spacer. Replacing this nucleotide from a T to a G lead an almost complete failure of transfected cells to activate GFP (FIG. 1F). To identify how the performance of the GFP-targeting crRNA is affected by the overall GC content of the upstream spacer in this array, the GC content of the spacer was varied. The HEK293T cells were transfected with these CRISPR arrays and a nuclease-deactivated Cas12a fused to the VP64-p65-Rta activator and mCherry (subsequently denoted dCas12a-VPR). Forty-eight hours later, the cells were analyzed by flow cytometry and quantified GFP fluorescence as a measure of array functionality.

Interestingly, a strong negative correlation between GC content of the spacer and GFP activation was observed (FIG. 1G). This indicated that crRNAs can exert a strong influence on the performance of the subsequent spacer, perhaps by forming secondary structures that interfere with Cas12a binding and processing.

Next, to analyze how the GC content varied over the length of these spacer sequences, all these random spacers were divided into three groups based on whether they enabled high, medium, or low GFP activation (FIG. 1G). For each spacer sequence, the GC content of a sliding 5-nt window was calculated and this sliding GC content was averaged for all spacers in the respective group (FIG. 1H). To get a closer view of the GC content very close to the cleavage site at the very 3′ end of the spacer, the size of the sliding window at the very 3′ end of the spacers was decreased. Interestingly, this analysis showed that particularly permissive spacers had a GC content that decreased toward the 3′ end of the spacer's length. In contrast, particularly non-permissive spacers had GC content that was slightly higher toward the 3′ end of the spacer. Spacers that enabled medium-level GFP activation had an overall quite high GC content but showed a drop in GC content close to the 3′ end of the spacer. This suggested that the importance of GC content was particularly high toward the 3′ end of the spacer, closer to the cleavage site where individual crRNAs are processed.

Surprisingly, spacers with a GC content in the 50-90% range displayed a wide spread of GFP activation, some enabling unexpectedly high GFP activation and others unexpectedly low (FIGS. 1G, 1I, and 1J). The sliding GC content of these spacers was analyzed, and an even stronger trend toward low GC content toward the 3′ end of the spacer for the unexpectedly permissive spacers and high GC content for the unexpectedly non-permissive spacers was found (FIGS. 1I-1J). Thus, even if the average GC content was high in a random spacer sequence, it could still allow efficient performance of the subsequent crRNA if GC content was low in the 3-5 bases at its very 3′ end. Conversely, even if a spacer's GC content was relatively low, it interfered with processing of the subsequent crRNA if the last few bases were of high GC content.

The GC content of the upstream spacer was moderately predictive of array performance (R²=0.45; FIG. 1K). The data suggested that the identity of the most 3′ bases might be disproportionately important for array performance. Indeed, it was found that approximately the same level of predictive power could be achieved by simply knowing the average GC content of the last three nucleotides in the upstream spacer (R²=0.52; FIG. 1K). These last three bases were more predictive of array performance than any other three bases in the spacer. These results suggested that optimal array performance requires approximately three bases immediately upstream of the Cas12a cleavage site to be As or Ts.

Further, computational analyses were performed to determine what impact, if any, secondary structure has on array performance. As demonstrated in FIG. 1L, the GC content of the upstream, non-targeting spacer is correlated with predicted secondary structure formation (estimated using the online tool RNAfold (Lorenz et al 2011. VIENNARNA package 2.0. Algorithms for Molecular Biology 6: 26. DOI: https://doi.org/10.1186/1748-7188-6-26, PMID: 22115189). Predicted secondary structure formation, in turn, is anticorrelated with performance of the GFP-targeting spacer (FIG. 1M), suggesting that strong secondary structures is what impedes array performance.

### Example 4: Separator Sequences With Low GC Content at the 3′ Ends

The purpose of this example is to demonstrate that separators play an important role during CRISPR array processing by providing an AT-rich sequence that gives Cas12a maximum accessibility to its cleavage site.

Whether bacteria have evolved mechanisms to incorporate only spacers with low GC content in their CRISPR arrays was investigated. To address this question, 727 naturally occurring Cas12a spacer sequences from 30 bacterial species were analyzed. However, no conspicuous absence of GC-rich spacers was found. Spacer GC content was normally distributed around an average of 39%, with a range of 10-70% (FIG. 2A). This was true also for Lachnospiraceae bacterium, the species from which the experimental dCas12a variant is derived. Neither was it found that GC content was lower at the 3′ end of these spacers (FIG. 2B). Therefore, the question of how Cas12a is able to process CRISPR arrays properly when some spacers might have very high GC content was investigated.

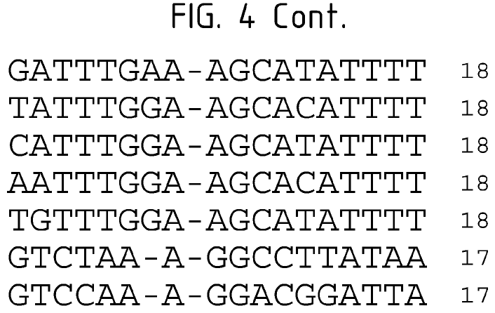

In naturally occurring CRISPR arrays, the separator sequence gets excised through the action of Cas12a and an unknown enzyme (FIG. 1B; Zetsche, Cell, 2015). Whether the separator might act as an insulator that protects every crRNA from disturbances caused by secondary structure in upstream spacers was investigated. To this end, 79 unique separator sequences from 30 bacterial species were analyzed (FIG. 4 and Table 3). Overall, these sequences showed very little sequence conservation (FIG. 2C). The only region that seemed to moderately evolutionarily conserved was a 5-nt sequence at the very 5′ end of the separator (GTYTA, SEQ ID NO: 30). Possibly this sequence acts as a binding site for the unknown enzyme responsible for cleaving its 5′ end during crRNA processing. However, despite there being so little sequence conservation, a strong bias for low GC content was detected (FIG. 2C). This suggests that the separator play an important role during CRISPR array processing by providing an AT-rich sequence that gives Cas12a maximum accessibility to its cleavage site.

TABLE 3
Separator sequences from 30 bacterial species

| Sequence | SEQ ID NO |
|---|---|
| GTCAAAAGACCTTTTT | 33 |
| GTCAAAAGGCCTTTTT | 34 |
| GTTTGAATAACCTTAAAT | 35 |
| GTTTGAATAGCCTTAAAT | 36 |
| GTTTGAATAATCTTAAAT | 37 |
| GTCTAAGAACTTTAAAT | 38 |
| CTCTAATAAGAGATATG | 39 |
| CTCTAATAGGAGATATG | 40 |
| CGCTAATAGGAGATATG | 41 |
| GTTTCAAAGATTAAAT | 42 |
| GTTTCAAAGATTGAAG | 43 |
| TTTTAAAAGATTGAAA | 44 |
| AGCTTAGAACATTTAAAA | 45 |
| TGCTTAGAACATTTAAAG | 46 |
| CTCTAAAGAGAGGAAAG | 47 |
| GTCTAACGACCTTTTA | 48 |
| CTCAAAACTCATTCG | 49 |
| GTTTAAAAGTCCTATTG | 50 |
| GCCAAATACCTCTATAA | 51 |
| GTCTAGGTACTCTCTTT | 52 |
| GTCAATAAGACTCATTT | 53 |
| ATCAATAAGACTCATTT | 54 |
| GCCTATAAGGCTTTAGT | 55 |
| AGCTATAAGGCTTTAGT | 56 |
| TGCTATAAGGCTTTAGT | 57 |
| GCCTATAAGGCTTCAGT | 58 |
| GTCCAAAGGACGGATTA | 59 |
| GTCTAAGACTTAAAGAT | 60 |
| GTCTAAGACTTAAAGTT | 61 |
| GTCTAAGACTTAAAGAAA | 62 |
| GTTTTAGAACCTTAAAAT | 63 |
| GTTTTAGAACCTTAAAA | 64 |
| GTTTTATAACCTTAAAAA | 65 |
| GTCTTAGAACCTTAAAA | 66 |
| GTTTTAGAACCTTTAAAA | 67 |
| GTCTAAGCCTTAGCTTA | 68 |
| GTTGAGACTGTAAGCGA | 69 |
| GTTGAAACTGTAAGCGG | 70 |
| GTCGAAACTGTAAGCGA | 71 |
| GTTAAAACTGTAAACGG | 72 |
| GTTGAAACCGTAAGCGG | 73 |
| GTTGAAACTGTAAAGAA | 74 |
| GTTGAAACTGTAAGAAA | 75 |
| GTTGAAACTGTGAGAAA | 76 |
| GTTAAGACTGCAAGGA | 77 |
| GTTAAAACTGTAAGCGG | 78 |
| ATTGAAACTGTAAAGA | 79 |
| GTCTGAAACTGTAAACGG | 80 |
| GTTGAAGCTGTAAGCAA | 81 |
| GTTGAATCTGTACGGA | 82 |
| GCTGAGATTGTAAAGTGA | 83 |
| GTTGGGACTGTGAGCCA | 84 |
| GTTGATACTGTGAGCGG | 85 |
| GTTGAAACTGTTAGGGG | 86 |
| GTAGACGATGAAGCGA | 87 |
| ATTGAGGCCGTAAGCAA | 88 |
| GTTTAAAACCACTTTAA | 89 |
| GTTAAATAATAAGAAAG | 90 |
| GTTAAATAATAAGAAAA | 91 |
| ATAAAATAATAAGAAAG | 92 |
| GTCTAACGACCTTCTA | 93 |
| GGCTACATAAAGCCTAT | 94 |
| GGCTACATAAAGCCTGT | 95 |
| TGCTACATAAAGCCTGT | 96 |
| GGCTACTTAAAGCCTAT | 97 |
| GCTTAGAACCTTTAAAT | 98 |
| GCTTAATCAACCCTTAG | 99 |
| GTTTAATCAACCCTTAG | 100 |
| GTTTAATAATCCTTTAG | 101 |
| GTCTAAAGGCCTTATAA | 102 |
| GATTTGAAAGCATCTTTT | 103 |
| TATTTGAAAGCATCTTTT | 104 |
| AATTTGAAAGCATCTTTT | 105 |
| AGTTTGAAAGCATATTTT | 106 |
| GATTTGAAAGCATATTTT | 107 |
| TATTTGGAAGCACATTTT | 108 |
| CATTTGGAAGCATATTTT | 109 |
| AATTTGGAAGCACATTTT | 110 |
| TGTTTGGAAGCATATTTT | 111 |

### Example 5: Including an Artificial Separator Sequence Between crRNAs Improves Array Performance in Human Cells

The purpose of this example is to demonstrate that including an artificial separator sequence between crRNAs improves array performance in human cells.

Whether CRISPR arrays would show improved performance in human cells if they included the full separator sequence between each crRNA was investigated. This hypothesis was tested using a similar experimental design as described previously, with a CRISPR array consisting of one crRNA containing a spacer, followed by a crRNA targeting the GFP promoter (FIG. 3A). The array either did or did not contain the natural separator sequence from L. bacterium. However, including this separator almost completely abolished array function, as nearly no GFP activation was seen in these cells (data not shown). This is consistent with a previous study that reported poor performance of crRNAs that contain the full-length, pre-processed Cas12a repeat (which contains the separator) (Liu et al., Nucleic Acids Research, 2019). One possible reason is that, because Cas12a cannot cleave the 5′ end of the separator, the separator will remain attached at the 3′ end of each processed crRNA. This might interfere with loading onto Cas12a or target DNA binding.

Then whether incorporating only parts of the separator would still retain its predicted insulating function was investigated. CRISPR arrays were generated in which the crRNAs were either separated by 1-4 nucleotides from the natural L. bacterium separator, or by a single G (FIGS. 3A-B). Three versions of each array were generated, where the GC content of the spacer was 30%, 50% or 70% (FIG. 3A). Interestingly, each addition of an A or T improved performance of the CRISPR array (FIG. 3C). These results suggested that this short, AT-rich artificial separator improved processing of the CRISPR array, despite each spacer now containing an AAAT sequence attached to their 3′ end.

Next, it was investigated whether the addition of this short, synthetic separator sequence would improve CRISPR activation of endogenous genes when crRNAs are expressed in a CRISPR array. For this, HEK293T cells were transfected (FIG. 1E) with a CRISPR array containing seven crRNAs (FIG. 3D). Each crRNA targeted the promoter of one gene, which included both protein-coding genes (CD9, IFNG, EGFR, TMEM107, GFP) and long non-coding RNAs (IMPACT, DANCR). The full length sequence of the construct, CAGp-BFP-Triplex-Leader-Array-Terminator, is provided in SEQ ID NO: 113.

(SEQ ID NO: 113)
GTGAGCCCCACGTTCTGCTTCACTCTCCCCATCTCCCCCCCCTCCCCACCCCCAAT
TTTGTATTTATTTATTTTTTAATTATTTTGTGCAGCGATGGGGGGGGGGGGGGGGG
GGGCGCGCGCCAGGCGGGGGGGGCGGGGCGAGGGGCGGGGCGGGGCGAGGCG
GAGAGGTGCGGCGGCAGCCAATCAGAGCGGCGCGCTCCGAAAGTTTCCTTTTAT
GGCGAGGCGGCGGCGGCGGCGGCCCTATAAAAAGCGAAGCGCGCGGCGGGCGG
GAGTCGCTGCGTTGCCTTCGCCCCGTGCCCCGCTCCGCGCCGCCTCGCGCCGCCC
GCCCCGGCTCTGACTGACCGCGTTACTCCCACAGGTGAGCGGGCGGGACGGCCC
TTCTCCTCCGGGCTGTAATTAGCGCTTGGTTTAATGACGGCTCGTTTCTTTTCTGT
GGCTGCGTGAAAGCCTTAAAGGGCTCCGGGAGGGCCCTTTGTGCGGGGGGGAGC
GGCTCGGGGGGTGCGTGCGTGTGTGTGTGCGTGGGGAGCGCCGCGTGCGGCCCG
CGCTGCCCGGCGGCTGTGAGCGCTGCGGGCGCGGCGCGGGGCTTTGTGCGCTCC
GCGTGTGCGCGAGGGGAGCGCGGCCGGGGGCGGTGCCCCGCGGTGCGGGGGGG
CTGCGAGGGGAACAAAGGCTGCGTGCGGGGTGTGTGCGTGGGGGGGTGAGCAG
GGGGTGTGGGCGCGGCGGTCGGGCTGTAACCCCCCCCTGCACCCCCCTCCCCGAG
TTGCTGAGCACGGCCCGGCTTCGGGTGCGGGGCTCCGTGCGGGGCGTGGCGCGG
GGCTCGCCGTGCCGGGCGGGGGGTGGCGGCAGGTGGGGGTGCCGGGCGGGGCG
GGGCCGCCTCGGGCCGGGGAGGGCTCGGGGGAGGGGCGCGGCGGCCCCGGAGC
GCCGGCGGCTGTCGAGGCGCGGCGAGCCGCAGCCATTGCCTTTTATGGTAATCGT
GCGAGAGGGCGCAGGGACTTCCTTTGTCCCAAATCTGGCGGAGCCGAAATCTGG
GAGGCGCCGCCGCACCCCCTCTAGCGGGCGCGGGCGAAGCGGTGCGGCGCCGGC
AGGAAGGAAATGGGCGGGGAGGGCCTTCGTGCGTCGCCGCGCCGCCGTCCCCTT
CTCCATCTCCAGCCTCGGGGCTGCCGCAGGGGGACGGCTGCCTTCGGGGGGGAC
GGGGCAGGGCGGGGTTCGGCTTCTGGCGTGTGACCGGCGGCTCTAGAGCCTCTG
CTAACCATGTTCATGCCTTCTTCTTTTTCCTACAGCTCCTGGGCAACGTGCTGGTT
GTTGTGCTGTCTCATCATTTTGGCAAAGAATTGAATTCGTCGCCACCATGGAGCTG
ATTAAGGAGAACATGCACATGAAGCTGTACATGGAGGGCACCGTGGACAACCATCACT
TCAAGTGCACATCCGAGGGCGAAGGCAAGCCCTACGAGGGCACCCAGACCATGAGAA
TCAAGGTGGTCGAGGGCGGCCCTCTCCCCTTCGCCTTCGACATCCTGGCTACTAGCTT
CCTCTACGGCAGCAAGACCTTCATCAACCACACCCAGGGCATCCCCGACTTCTTCAAG
CAGTCCTTCCCTGAGGGCTTCACATGGGAGAGAGTCACCACATACGAAGACGGGGGC
GTGCTGACCGCTACCCAGGACACCAGCCTCCAGGACGGCTGCCTCATCTACAACGTC
AAGATCAGAGGGGTGAACTTCACATCCAACGGCCCTGTGATGCAGAAGAAAACACTCG
GCTGGGAGGCCTTCACCGAGACGCTGTACCCCGCTGACGGCGGCCTGGAAGGCAGA
AACGACATGGCCCTGAAGCTCGTGGGGGGAGCCATCTGATCGCAAACATCAAGACC
ACATATAGATCCAAGAAACCCGCTAAGAACCTCAAGATGCCTGGCGTCTACTATGTGGA
CTACAGACTGGAAAGAATCAAGGAGGCCAACAACGAGACCTACGTCGAGCAGCACGA
GGTGGCAGTGGCCAGATACTGCGACCTCCCTAGCAAACTGGGGCACAAGCTTAATTA

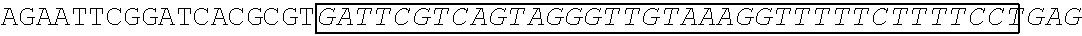

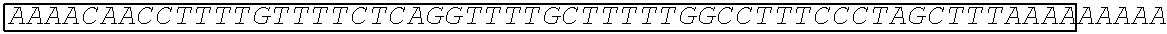

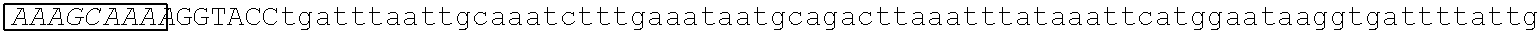

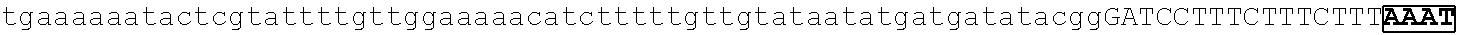

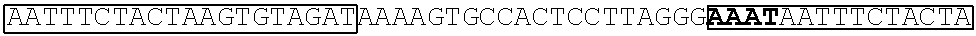

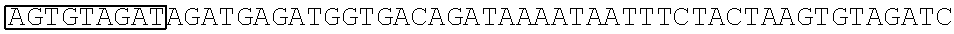

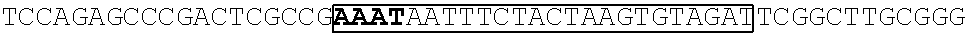

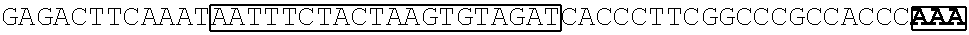

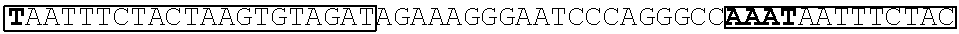

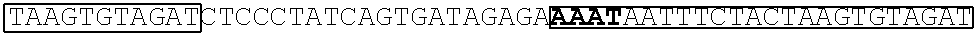

GTTAACTTGTTTATTGCAGCTTATAATGGTTACAAATAAAGCAATAGCATCACAAATTTC
ACAAATAAAGCATTTTTTTCACTGCATTCTAGTTGTGGTTTGTCCAAACTCATCAATGTAT
CTTATCATGTCTGGATC

From 5′-to 3′-end, the CAG promoter sequence is double-underlined. The BFP sequence is italicized. The triplex sequence is italicized and boxed. Each of the seven CRISPR array sequences is boxed. Six of the 7 CRISPR array sequences have a separator sequence AAAT, which is bolded. The Lachnospiraceae bacterium leader sequence is in small letters. And the SV40 terminator sequence is on the 3′-terminus, double underlined and italicized.

The seven target genes were selected partly because of their different baseline expression levels in HEK293 cells (Hagemann-Jensen et al., Nature Biotech, 2020). Results showed that including the synthetic AAAT separator increased activation levels of all target genes compared to the array lacking the AAAT separator (FIG. 3E). The effect size was modest (ranging from 1.1-fold to 8.0-fold), but consistent for all target genes (FIG. 3F). This increase was also seen on the protein level, which we could analyze for GFP (FIG. 3G). These results indicated that including a short, AT-rich separator sequence between each crRNA in a Cas12a CRISPR array increases the efficacy of CRISPR-activation.

The use of artificial separators derived from multiple bacterial species was also examined for the ability to rescue poor GFP activation caused by a non-permissive non-targeting dummy spacer upstream of the targeting spacer in a CRISPR array (FIG. 3H).

Further, the enhanced Cas12a protein from Acidaminococcus species was also shown to be sensitive to GC content of an upstream non-targeting dummy spacer (FIG. 3I). Its performance can be rescued using a TTTT synSeparator derived from its natural separator (FIG. 3I).

### Example 6: A Cas13d/Cas12a CRISPR Hybrid Array Enables Simultaneous Upregulation and Downregulation of Different Genes

The purpose of this example is to demonstrate that a Cas12/Cas13 CRISPR hybrid array can be used to simultaneously up- and downregulate genes in cells.

Whether a single CRISPR hybrid array can be used to upregulate some genes while simultaneously downregulating other genes was tested in this experiment. This hypothesis was tested using a similar experimental setup as described previously, but using HEK293T cells carrying both genomically integrated GFP driven by the TRE3G promoter and a genomically integrated Tre3G gene driven by the EF1a promoter. A CRISPR array was used containing two Cas13d gRNAs targeting GFP mRNA and one Cas12a gRNA targeting the CD9 promoter. Cells were transfected with the CRISPR hybrid arrays and a dCas12a-miniVPR activator and Cas13d. Cells transfected with all three constructs were stained with a CD9-targeting antibody and analyzed using flow cytometry to measure APC fluorescence and GFP fluorescence. These cells show simultaneous upregulation of CD9 and downregulation of GFP (FIG. 7C) demonstrating that a single CRISPR hybrid array can specify some genes for upregulation by one protein and other genes for downregulation by another Cas protein.

The full length sequence of the construct, used in FIG. 7C, is provided in SEQ ID NO: 117.

(SEQ ID NO: 117)
GTGAGCCCCACGTTCTGCTTCACTCTCCCCATCTCCCCCCCCTCCCCACCCCCAAT
TTTGTATTTATTTATTTTTTAATTATTTTGTGCAGCGATGGGGGCGGGGGGGGGGG
GGGCGCGCGCCAGGCGGGGCGGGGCGGGGCGAGGGGCGGGGCGGGGCGAGGCG
GAGAGGTGCGGCGGCAGCCAATCAGAGCGGCGCGCTCCGAAAGTTTCCTTTTAT
GGCGAGGCGGCGGCGGCGGCGGCCCTATAAAAAGCGAAGCGCGCGGCGGGCGG
GAGTCGCTGCGTTGCCTTCGCCCCGTGCCCCGCTCCGCGCCGCCTCGCGCCGCCC
GCCCCGGCTCTGACTGACCGCGTTACTCCCACAGGTGAGCGGGCGGGACGGCCC
TTCTCCTCCGGGCTGTAATTAGCGCTTGGTTTAATGACGGCTCGTTTCTTTTCTGT
GGCTGCGTGAAAGCCTTAAAGGGCTCCGGGAGGGCCCTTTGTGCGGGGGGGAGC
GGCTCGGGGGGTGCGTGCGTGTGTGTGTGCGTGGGGAGCGCCGCGTGCGGCCCG
CGCTGCCCGGCGGCTGTGAGCGCTGCGGGCGCGGCGCGGGGCTTTGTGCGCTCC
GCGTGTGCGCGAGGGGAGCGCGGCCGGGGGCGGTGCCCCGCGGTGCGGGGGGG
CTGCGAGGGGAACAAAGGCTGCGTGCGGGGTGTGTGCGTGGGGGGGTGAGCAG
GGGGTGTGGGCGCGGCGGTCGGGCTGTAACCCCCCCCTGCACCCCCCTCCCCGAG
TTGCTGAGCACGGCCCGGCTTCGGGTGCGGGGCTCCGTGCGGGGCGTGGCGCGG
GGCTCGCCGTGCCGGGCGGGGGGTGGCGGCAGGTGGGGGTGCCGGGCGGGGCG
GGGCCGCCTCGGGCCGGGGAGGGCTCGGGGGAGGGGCGCGGCGGCCCCGGAGC
GCCGGCGGCTGTCGAGGCGCGGCGAGCCGCAGCCATTGCCTTTTATGGTAATCGT
GCGAGAGGGCGCAGGGACTTCCTTTGTCCCAAATCTGGCGGAGCCGAAATCTGG
GAGGCGCCGCCGCACCCCCTCTAGCGGGCGCGGGCGAAGCGGTGCGGCGCCGGC
AGGAAGGAAATGGGCGGGGAGGGCCTTCGTGCGTCGCCGCGCCGCCGTCCCCTT
CTCCATCTCCAGCCTCGGGGCTGCCGCAGGGGGACGGCTGCCTTCGGGGGGGAC
GGGGCAGGGCGGGGTTCGGCTTCTGGCGTGTGACCGGCGGctctagagcctctgctaaccatgtt
catgccttcttctttttcctacagctcctgggcaacgtgctggttgttgtgctgtctcatcattttggcaaagaattgaattcgtcgccaccAT
GGAGCTGATTAAGGAGAACATGCACATGAAGCTGTACATGGAGGGCACCGTGGACAA
CCATCACTTCAAGTGCACATCCGAGGGCGAAGGCAAGCCCTACGAGGGCACCCAGAC
CATGAGAATCAAGGTGGTCGAGGGCGGCCCTCTCCCCTTCGCCTTCGACATCCTGGC
TACTAGCTTCCTCTACGGCAGCAAGACCTTCATCAACCACACCCAGGGCATCCCCGAC
TTCTTCAAGCAGTCCTTCCCTGAGGGCTTCACATGGGAGAGAGTCACCACATACGAAG
ACGGGGGCGTGCTGACCGCTACCCAGGACACCAGCCTCCAGGACGGCTGCCTCATCT
ACAACGTCAAGATCAGAGGGGTGAACTTCACATCCAACGGCCCTGTGATGCAGAAGAA
AACACTCGGCTGGGAGGCCTTCACCGAGACGCTGTACCCCGCTGACGGCGGCCTGGA
AGGCAGAAACGACATGGCCCTGAAGCTCGTGGGCGGGAGCCATCTGATCGCAAACAT
CAAGACCACATATAGATCCAAGAAACCCGCTAAGAACCTCAAGATGCCTGGCGTCTACT
ATGTGGACTACAGACTGGAAAGAATCAAGGAGGCCAACAACGAGACCTACGTCGAGCA
GCACGAGGTGGCAGTGGCCAGATACTGCGACCTCCCTAGCAAACTGGGGCACAAGCT

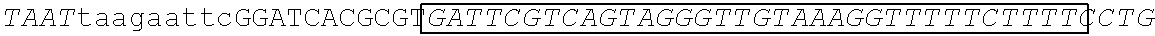

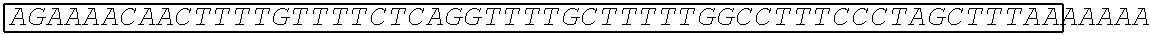

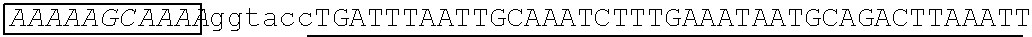

TATAAATTCATGGAATAAGGTGATTTTATTGTGAAAAAATACTCGTATTTTGTTG
GAAAAACATCTTTTTGTTGTATAATATGATGATATACGGGATCCTTTCTTTCAAG
TAAACCCCTACCAACTGGTCGGGGTTTGAAACggtgctcaggtagtggttgtcgggAAATA
ATTTCTACTAAGTGTAGATaaaagtgccactccttagggCAAGTAAACCCCTACCAACTG

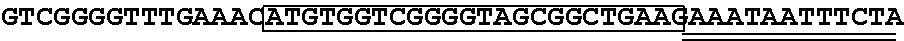

CTAAGTGTAGATgttaacTTGTTTATTGCAGCTTATAATGGTTACAAATAAAGCAATAGCA
TCACAAATTTCACAAATAAAGCATTTTTTTCACTGCATTCTAGTTGTGGTTTGTCCAAACT
CATCAATGTATCTTATCATGTCTGGATC

From 5′-to 3′-end, the CAG promoter sequence is double-underlined. The BFP sequence is italicized. The triplex sequence is italicized and boxed. Each of the seven CRISPR array sequences is boxed. The Lachnospiraceae bacterium leader sequence is bold underlined. And the SV40 terminator sequence is on the 3′-terminus, double underlined and italicized. The Cas13d CRISPR-repeat sequence is bolded. The Cas12a CRISPR-repeat is bolded and double underlined. The GFP-targeting Cas13d spacer #1 is in lowercase italics. The CD9-targeting Cas12a spacer is lowercase underlined. The GFP-targeting Cas13d spacer #1 is bolded and boxed.

Next, whether the order of Cas13d and Cas12a gRNAs on the single CRISPR hybrid array matters for gene modulation efficacy was examined. The same experimental setup was used as in FIG. 7C. CRISPR arrays were designed that carried three Cas12a gRNAs for gene upregulation and three Cas13d gRNAs for gene downregulation. Some array designs contained a triplex sequence between the Cas12a and Cas13d gRNAs. The triplex sequence forms a stabilizing RNA secondary structure that stabilizes the upstream transcript (Campa et al., Nature Methods, 2019). The CRISPR arrays contained six gRNAs, but in this experiment only two target genes were measured (CD9 and GFP) to assess the performance of each array design. These constructs were used in FIG. 7D. For FIG. 7D, the sequences upstream of the downstream of the CRISPR arrays were identical to the above sequence SEQ ID NO:117 ((i.e., the sequence ending with ...ATCCTTTCTTT, and the sequence starting with gttaacttgtttatt...). The CRISPR arrays themselves had the following sequences: Design A construct, used in FIG. 7D, is provided in SEQ ID NO: 118 as follows:

(SEQ ID NO: 118)
CAAGTAAACCCCTACCAACTGGTCGGGGTTTGAAACATGTGGTCGGGGTAGCGGCTG

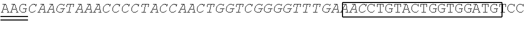

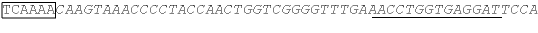

GTCGCTGTCAAATAATTTCTACTAAGTGTAGATaaaagtgccactccttagggAAATAATTT

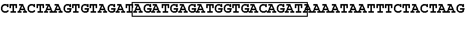

TGTAGATcaggagggtgactcaggctaAAATAATTTCTACTAAGTGTAGAT

The Cas13d CRISPR-repeat sequence is in italics. The Cas12a CRISPR-repeat sequence is bolded. The GFP-targeting Cas13d spacer sequence is double underlined. The HRAS-targeting Cas13d spacer is boxed. The SMARCA4-targeting Cas13d spacer sequence is italicized and underlined. The CD9-targeting Cas12a spacer sequence is lowercase. The IFNG-targeting Cas12a spacer sequence is bolded and boxed. The IL1RN-targeting Cas12a spacer sequence is italicized in lowercase.

The Design B construct, used in FIG. 7D, is provided in SEQ ID NO: 119 as follows:

CAAGTAAACCCCTACCAACTGGTCGGGGTTTGAAACATGTGGTCGGGGTAGCGGCTG

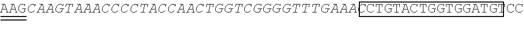

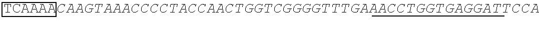

GTCGCTGTCCAAGTAAACCCCTACCAACTGGTCGGGGTTTGAAACGATTCGTCAGTA
GGGTTGTAAAGGTTTTTCTTTTCCTGAGAAAACAACCTTTTGTTTTCTCAGGT
TTTGCTTTTTGGCCTTTCCCTAGCTTTAAAAAAAAAAAAGCAAAAAAATAATT
TCTACTAAGTGTAGATaaaagtgccactccttagggAAATAATTTCTACTAAGTGTAGAT

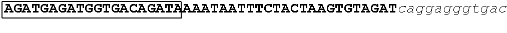

tcaggctaAAATAATTTCTACTAAGTGTAGAT

The Cas13d CRISPR-repeat sequence is in italics. The Cas12a CRISPR-repeat sequence is bolded. The GFP-targeting Cas13d spacer sequence is double underlined. The HRAS-targeting Cas13d spacer is boxed. The SMARCA4-targeting Cas13d spacer sequence is italicized and underlined. The CD9-targeting Cas12a spacer sequence is lowercase. The IFNG-targeting Cas12a spacer sequence is bolded and boxed. The IL1RN-targeting Cas12a spacer sequence is italicized in lowercase. The triplex sequence is bolded and underlined.

The Design C construct, used in FIG. 7D, is provided in SEQ ID NO: 120 as follows:

AAATAATTTCTACTAAGTGTAGATaaaagtgccactccttagggAAATAATTTCTACTAAG

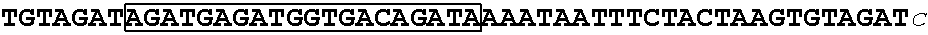

aggagggtgactcaggctaCAAGTAAACCCCTACCAACTGGTCGGGGTTTGAAACATGTGGTC

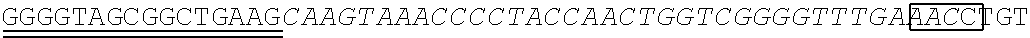

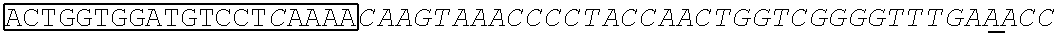

TGGTGAGGATTCCAGTCGCTGTCCAAGTAAACCCCTACCAACTGGTCGGGGTTTGAAA
C

The Cas13d CRISPR-repeat sequence is in italics. The Cas12a CRISPR-repeat sequence is bolded. The GFP-targeting Cas13d spacer sequence is double underlined. The HRAS-targeting Cas13d spacer is boxed. The SMARCA4-targeting Cas13d spacer sequence is italicized and underlined. The CD9-targeting Cas12a spacer sequence is lowercase. The IFNG-targeting Cas12a spacer sequence is bolded and boxed. The IL1RN-targeting Cas12a spacer sequence is italicized in lowercase.

The Design D construct, used in FIG. 7D, is provided in SEQ ID NO: 121 as follows:

AAATAATTTCTACTAAGTGTAGATaaaagtgccactccttagggAAATAATTTCTACTAAG

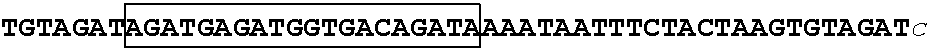

aggagggtgactcaggctaAAATAATTTCTACTAAGTGTAGATGATTCGTCAGTAGGGT
TGTAAAGGTTTTTCTTTTCCTGAGAAAACAACCTTTTGTTTTCTCAGGTTTTG
CTTTTTGGCCTTTCCCTAGCTTTAAAAAAAAAAAAGCAAAACAAGTAAACCCCT
ACCAACTGGTCGGGGTTTGAAACATGTGGTCGGGGTAGCGGCTGAAGCAAGTAAAC

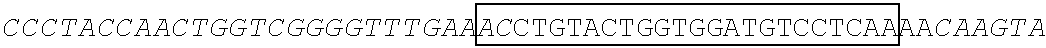

AACCCCTACCAACTGGTCGGGGTTTGAAACCTGGTGAGGATTCCAGTCGCTGTCCAAG
TAAACCCCTACCAACTGGTCGGGGTTTGAAAC

The Cas13d CRISPR-repeat sequence is in italics. The Cas12a CRISPR-repeat sequence is bolded. The GFP-targeting Cas13d spacer sequence is double underlined. The HRAS-targeting Cas13d spacer is boxed. The SMARCA4-targeting Cas13d spacer sequence is italicized and underlined. The CD9-targeting Cas12a spacer sequence is lowercase. The IFNG-targeting Cas12a spacer sequence is bolded and boxed. The IL1RN-targeting Cas12a spacer sequence is italicized in lowercase. The triplex sequence is bolded and underlined.

The Design M construct, used in FIG. 7D, is provided in SEQ ID NO: 122 as follows:

CAAGTAAACCCCTACCAACTGGTCGGGGTTTGAAACATGTGGTCGGGGTAGCGGCTG
AAGAAATAATTTCTACTAAGTGTAGATaaaagtgccactccttagggCAAGTAAACCCCTAC

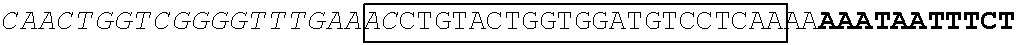

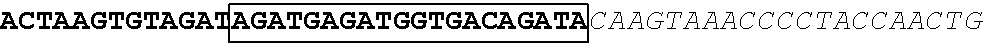

GTCGGGGTTTGAAACCTGGTGAGGATTCCAGTCGCTGTCAAATAATTTCTACTAAG
TGTAGATcaggagggtgactcaggctaCAAGTAAACCCCTACCAACTGGTCGGGGTTTGAAAC

The Cas13d CRISPR-repeat sequence is in italics. The Cas12a CRISPR-repeat sequence is bolded. The GFP-targeting Cas13d spacer sequence is double underlined. The HRAS-targeting Cas13d spacer is boxed. The SMARCA4-targeting Cas13d spacer sequence is italicized and underlined. The CD9-targeting Cas12a spacer sequence is lowercase. The IFNG-targeting Cas12a spacer sequence is bolded and boxed. The IL1RN-targeting Cas12a spacer sequence is italicized in lowercase.

Two days after transfection, cells were prepared for flow cytometry as described in the methods above. The experiment showed that all array designs led to simultaneous upregulation of CD9 by dCas12a-miniVPR and downregulation of GFP by Cas13d (FIG. 7D). These results demonstrate that various designs for CRISPR hybrid arrays can be used for simultaneous up- and downregulation of genes.

While particular alternatives of the present disclosure have been disclosed, it is to be understood that various modifications and combinations are possible and are contemplated within the true spirit and scope of the appended claims. There is no intention, therefore, of limitations to the exact abstract and disclosure herein presented.

What is claimed is:

1. An engineered multiplex Cluster Regularly Interspaced Short Palindromic Repeat (CRISPR) array comprising more than one CRISPR RNA (crRNA), wherein each of the more than one crRNAs comprises a repeat sequence and a spacer, wherein the spacer is configured to hybridize to a specific target nucleic acid of a plurality of target nucleic acids, and wherein the repeat sequence in each of the more than one crRNAs is preceded by a separator sequence.

2. The engineered multiplex CRISPR array of claim 1, wherein at least a portion of the more than one crRNAs comprise a Cas12a repeat sequence.

3. The engineered multiplex CRISPR array of claim 2, wherein the engineered multiplex CRISPR array is capable of upregulating the expression of the plurality of target nucleic acids simultaneously.

4. The engineered multiplex CRISPR array of claim 1, wherein at least a portion of the more than one crRNAs comprise a Cas13 repeat sequence.

5. The engineered multiplex CRISPR array of claim 4, wherein the engineered multiplex CRISPR array is capable of downregulating the expression of the plurality of target nucleic acids simultaneously.

6. The engineered multiplex CRISPR array of claim 1, wherein at least a portion of the more than one crRNAs comprise a Cas12a repeat sequence and at least a portion of the more than one crRNAs comprise a Cas13 repeat sequence.

7. The engineered multiplex CRISPR array of claim 6, wherein the engineered multiplex CRISPR array is capable of upregulating and downregulating the expression of the plurality of target nucleic acids simultaneously.

8. The engineered multiplex CRISPR array of any one of the preceding claims, wherein the plurality of target nucleic acids comprises at least 4 different target nucleic acids.

9. The engineered multiplex CRISPR array of any one of the preceding claims, wherein the Cas13 protein comprises a Cas13d protein and a Cas13b protein.

10. The engineered multiplex CRISPR array of any one of the preceding claims, wherein the average length of the crRNA is about 30 to about 70 nucleotides.

11. The engineered multiplex CRISPR array of any one of the preceding claims, wherein the average length of the crRNA is about 50 nucleotides.

12. The engineered multiplex CRISPR array of any one of the preceding claims, wherein the separator sequence comprises an AT-rich sequence, and wherein the separator sequence is about 3 to about 8 nucleotides in length.

13. The engineered multiplex CRISPR array of any one of the preceding claims, wherein the plurality of target nucleic acids are RNAs.

14. The engineered multiplex CRISPR array of any one of the preceding claims, wherein the plurality of target nucleic acids are double-stranded DNAs (dsDNAs).

15. A nucleic acid encoding the engineered multiplex CRISPR array of any one of the preceding claims.

16. A vector comprising the nucleic acid of claim 15.

17. The vector of claim 16, further comprising a promoter.

18. The vector of claim 16 or 17, wherein the promoter comprises a polymerase II promoter.

19. The vector of any one of claims 16-18, wherein the polymerase II promoter comprises a CAG promoter, an avPGK promoter, an EF1a promoter, and a SFFV promoter.

20. The vector of any one of claims 16-19, further comprising a reporter gene.

21. The vector of any one of claims 16-20, wherein the reporter gene comprises BFP, GFP, and mCherry.

22. The vector of any one of claims 16-21, wherein the vector comprises a lentiviral vector, Adeno-associated viral vector, and piggyBac vector.

23. A method of making a collection of engineered multiplex CRISPR arrays, comprising:
providing more than one crRNAs, wherein each of the more than one crRNAs comprises a 5′ oligonucleotide overhang and a 3′ oligonucleotide overhang configured to hybridize to each other; wherein each of the more than one crRNAs comprises a repeat sequence and a spacer, wherein the spacer is configured to hybridize to a specific target nucleic acid of a plurality of target nucleic acids, and wherein the repeat sequence in each of the more than one crRNAs is preceded by a separator sequence; and
randomly hybridizing the more than one crRNAs to generate the collection of the engineered multiplex CRISPR arrays.

24. The method of claim 23, wherein the repeat sequences in the more than one crRNAs comprise Cas12a repeat sequence, a Cas13 repeat sequence, or both Cas12a and Cas13 repeat sequences.

25. The method of claim 23 or 24, wherein the Cas13 repeat sequence comprises a Cas13d repeat sequence and a Cas13b repeat sequence.

26. The method of any one of claims 23-25, wherein the collection of the engineered multiplex CRISPR arrays is capable of upregulating and downregulating the expression of the plurality of target nucleic acids simultaneously.

27. The method of any one of claims 23-26, wherein the plurality of target nucleic acids comprises at least 4 different target nucleic acids.

28. The method of any one of claims 23-27, wherein the average length of the crRNA is about 30 to about 70 nucleotides.

29. The method of any one of claims 23-28, wherein the average length of the crRNA is about 50 nucleotides.

30. The method of any one of claims 23-29, wherein the spacer comprises an A or an T at the 3′ end.

31. The method of any one of claims 23-30, wherein the separator sequence comprises an AT-rich linker sequence.

32. The method of any one of claims 23-31, wherein the separator sequence is about 3 to about 8 nucleotides in length.

33. The method of any one of claims 23-32, further comprising identifying the collection of engineered multiplex CRISPR arrays having a desired length.

34. The method of any one of claims 23-33, further comprising inserting the collection of the engineered multiplex CRISPR arrays into a vector.

35. The method of any one of claims 23-34, wherein the vector comprises a eukaryotic expression vector.

36. The method of any one of claims 23-35, further comprising delivering the collection of the engineered multiplex CRISPR arrays into host cells.

37. The method of any one of claims 23-36, wherein the host cells express the more than one Cas proteins.

38. The method of any one of claims 23-37, further comprising screening for the collection of engineered multiplex CRISPR arrays with a desired phenotype.

39. The method of any one of claims 23-38, wherein the screening comprises isolating the host cells exhibiting the desired phenotype.

40. The method of any one of claims 23-39, wherein the screening further comprises sequencing the engineered multiplex CRISPR array expressed by the isolated host cells.

41. The method of any one of claims 23-40, wherein the desired phenotype comprises controlled stem cell differentiation, controlled killing of tumor cells, and enhanced cell proliferation, increased T-cell activity level, and modified metabolic activity.

42. A method for simultaneous upregulation of multiple endogenous genes, comprising contacting a host cell with the engineered multiplex CRISPR array of any one of claims 1-14, wherein the more than one crRNAs comprise Cas12a repeat sequences and spacers configured to hybridize to a plurality of target nucleic acids.

43. A method for simultaneous downregulation of multiple endogenous genes, comprising contacting a host cell with the engineered multiplex CRISPR array of any one of claims 1-14, wherein the more than one crRNAs comprise Cas13 repeat sequences and spacers configured to hybridize to a plurality of target nucleic acids.

44. A method for simultaneous upregulation and downregulation of multiple endogenous genes, comprising contacting a host cell with the engineered multiplex CRISPR array of any one of claims 1-14, wherein the more than one crRNAs comprise both Cas12a and Cas13 repeat sequences and spacers configured to hybridize to a plurality of target nucleic acids.

45. The method of any one of claims 42-44, wherein the host cell expresses Cas12a proteins, Cas13 proteins, or both Cas12a proteins and Cas13 proteins.


SEQUENCE LISTING (122 sequences)

SEQ ID NO: 1 - DNA, 20 bp, Artificial sequence
  Synthetic construct
aatttctactaagtgtagat

SEQ ID NO: 2 - DNA, 36 bp, Artificial sequence
  Synthetic construct
caagtaaacccctaccaactggtcggggtttgaaac

SEQ ID NO: 3 - DNA, 4 bp, Artificial sequence
  Synthetic construct
aaat

SEQ ID NO: 4 - DNA, 5 bp, Artificial sequence
  Synthetic construct
ttata

SEQ ID NO: 5 - DNA, 5 bp, Artificial sequence
  Synthetic construct
attaa

SEQ ID NO: 6 - DNA, 7 bp, Artificial sequence
  Synthetic construct
tataatt

SEQ ID NO: 7 - DNA, 23 bp, Artificial sequence
  Synthetic construct
cctggaggagaagaggaaagaga

SEQ ID NO: 8 - DNA, 25 bp, Artificial sequence
  Synthetic construct
ttgaggacctctgtgtatttgtcaa

SEQ ID NO: 9 - DNA, 22 bp, Artificial sequence
  Synthetic construct
caccaagtgcatcaaatacctg

SEQ ID NO: 10 - DNA, 21 bp, Artificial sequence
  Synthetic construct
gtttcttgctcgaagatgctc

SEQ ID NO: 11 - DNA, 23 bp, Artificial sequence
  Synthetic construct
tcggtaactgacttgaatgtcca

SEQ ID NO: 12 - DNA, 21 bp, Artificial sequence
  Synthetic construct
tcgcttccctgttttagctgc

SEQ ID NO: 13 - DNA, 20 bp, Artificial sequence
  Synthetic construct
ttgccgcaaagtgtgtaacg

SEQ ID NO: 14 - DNA, 20 bp, Artificial sequence
  Synthetic construct
gtcacccctaaatgccaccg

SEQ ID NO: 15 - DNA, 21 bp, Artificial sequence
  Synthetic construct
gagtctccatgttcaacagca

SEQ ID NO: 16 - DNA, 21 bp, Artificial sequence
  Synthetic construct
tcccaacgctcgaatatgaag

SEQ ID NO: 17 - DNA, 21 bp, Artificial sequence
  Synthetic construct
tacagctccacctatctacca

SEQ ID NO: 18 - DNA, 23 bp, Artificial sequence
  Synthetic construct
acatctcttattttctccaccca

SEQ ID NO: 19 - DNA, 21 bp, Artificial sequence
  Synthetic construct
aggagttcgtctcttacgtct

SEQ ID NO: 20 - DNA, 21 bp, Artificial sequence
  Synthetic construct
tgaaataccagcaacaggaca

SEQ ID NO: 21 - DNA, 19 bp, Artificial sequence
  Synthetic construct
acttcaagagcgccatcca

SEQ ID NO: 22 - DNA, 20 bp, Artificial sequence
  Synthetic construct
gtcttgaaggcgtgctggta

SEQ ID NO: 23 - DNA, 20 bp, Artificial sequence
  Synthetic construct
aaaagtgccactccttaggg

SEQ ID NO: 24 - DNA, 20 bp, Artificial sequence
  Synthetic construct
agatgagatggtgacagata

SEQ ID NO: 25 - DNA, 20 bp, Artificial sequence
  Synthetic construct
ctccagagcccgactcgccg

SEQ ID NO: 26 - DNA, 20 bp, Artificial sequence
  Synthetic construct
tcggcttgcggggagacttc

SEQ ID NO: 27 - DNA, 20 bp, Artificial sequence
  Synthetic construct
cacccttcggcccgccaccc

SEQ ID NO: 28 - DNA, 20 bp, Artificial sequence
  Synthetic construct
agaaagggaatcccagggcc

SEQ ID NO: 29 - DNA, 20 bp, Artificial sequence
  Synthetic construct
ctccctatcagtgatagaga

SEQ ID NO: 30 - DNA, 5 bp, Artificial sequence
  Synthetic construct
gtyta

SEQ ID NO: 31 - DNA, 21 bp, Artificial sequence
  Synthetic construct
cgccaaacgtgccctgacggt

SEQ ID NO: 32 - DNA, 21 bp, Artificial sequence
  Synthetic construct
cgccaaacgtgccctgacggg

SEQ ID NO: 33 - DNA, 16 bp, Artificial sequence
  Synthetic construct
gtcaaaagaccttttt

SEQ ID NO: 34 - DNA, 16 bp, Artificial sequence
  Synthetic construct
gtcaaaaggccttttt

SEQ ID NO: 35 - DNA, 18 bp, Artificial sequence
  Synthetic construct
gtttgaataaccttaaat

SEQ ID NO: 36 - DNA, 18 bp, Artificial sequence
  Synthetic construct
gtttgaatagccttaaat

SEQ ID NO: 37 - DNA, 18 bp, Artificial sequence
  Synthetic construct
gtttgaataatcttaaat

SEQ ID NO: 38 - DNA, 17 bp, Artificial sequence
  Synthetic construct
gtctaagaactttaaat

SEQ ID NO: 39 - DNA, 17 bp, Artificial sequence
  Synthetic construct
ctctaataagagatatg

SEQ ID NO: 40 - DNA, 17 bp, Artificial sequence
  Synthetic construct
ctctaataggagatatg

SEQ ID NO: 41 - DNA, 17 bp, Artificial sequence
  Synthetic construct
cgctaataggagatatg

SEQ ID NO: 42 - DNA, 16 bp, Artificial sequence
  Synthetic construct
gtttcaaagattaaat

SEQ ID NO: 43 - DNA, 16 bp, Artificial sequence
  Synthetic construct
gtttcaaagattgaag

SEQ ID NO: 44 - DNA, 16 bp, Artificial sequence
  Synthetic construct
ttttaaaagattgaaa

SEQ ID NO: 45 - DNA, 18 bp, Artificial sequence
  Synthetic construct
agcttagaacatttaaaa

SEQ ID NO: 46 - DNA, 18 bp, Artificial sequence
  Synthetic construct
tgcttagaacatttaaag

SEQ ID NO: 47 - DNA, 17 bp, Artificial sequence
  Synthetic construct
ctctaaagagaggaaag

SEQ ID NO: 48 - DNA, 16 bp, Artificial sequence
  Synthetic construct
gtctaacgacctttta

SEQ ID NO: 49 - DNA, 15 bp, Artificial sequence
  Synthetic construct
ctcaaaactcattcg

SEQ ID NO: 50 - DNA, 17 bp, Artificial sequence
  Synthetic construct
gtttaaaagtcctattg

SEQ ID NO: 51 - DNA, 17 bp, Artificial sequence
  Synthetic construct
gccaaatacctctataa

SEQ ID NO: 52 - DNA, 17 bp, Artificial sequence
  Synthetic construct
gtctaggtactctcttt

SEQ ID NO: 53 - DNA, 17 bp, Artificial sequence
  Synthetic construct
gtcaataagactcattt

SEQ ID NO: 54 - DNA, 17 bp, Artificial sequence
  Synthetic construct
atcaataagactcattt

SEQ ID NO: 55 - DNA, 17 bp, Artificial sequence
  Synthetic construct
gcctataaggctttagt

SEQ ID NO: 56 - DNA, 17 bp, Artificial sequence
  Synthetic construct
agctataaggctttagt

SEQ ID NO: 57 - DNA, 17 bp, Artificial sequence
  Synthetic construct
tgctataaggctttagt

SEQ ID NO: 58 - DNA, 17 bp, Artificial sequence
  Synthetic construct
gcctataaggcttcagt

SEQ ID NO: 59 - DNA, 17 bp, Artificial sequence
  Synthetic construct
gtccaaaggacggatta

SEQ ID NO: 60 - DNA, 17 bp, Artificial sequence
  Synthetic construct
gtctaagacttaaagat

SEQ ID NO: 61 - DNA, 17 bp, Artificial sequence
  Synthetic construct
gtctaagacttaaagtt

SEQ ID NO: 62 - DNA, 18 bp, Artificial sequence
  Synthetic construct
gtctaagacttaaagaaa

SEQ ID NO: 63 - DNA, 18 bp, Artificial sequence
  Synthetic construct
gttttagaaccttaaaat

SEQ ID NO: 64 - DNA, 17 bp, Artificial sequence
  Synthetic construct
gttttagaaccttaaaa

SEQ ID NO: 65 - DNA, 18 bp, Artificial sequence
  Synthetic construct
gttttataaccttaaaaa

SEQ ID NO: 66 - DNA, 17 bp, Artificial sequence
  Synthetic construct
gtcttagaaccttaaaa

SEQ ID NO: 67 - DNA, 18 bp, Artificial sequence
  Synthetic construct
gttttagaacctttaaaa

SEQ ID NO: 68 - DNA, 17 bp, Artificial sequence
  Synthetic construct
gtctaagccttagctta

SEQ ID NO: 69 - DNA, 17 bp, Artificial sequence
  Synthetic construct
gttgagactgtaagcga

SEQ ID NO: 70 - DNA, 17 bp, Artificial sequence
  Synthetic construct
gttgaaactgtaagcgg

SEQ ID NO: 71 - DNA, 17 bp, Artificial sequence
  Synthetic construct
gtcgaaactgtaagcga

SEQ ID NO: 72 - DNA, 17 bp, Artificial sequence
  Synthetic construct
gttaaaactgtaaacgg

SEQ ID NO: 73 - DNA, 17 bp, Artificial sequence
  Synthetic construct
gttgaaaccgtaagcgg

SEQ ID NO: 74 - DNA, 17 bp, Artificial sequence
  Synthetic construct
gttgaaactgtaaagaa

SEQ ID NO: 75 - DNA, 17 bp, Artificial sequence
  Synthetic construct
gttgaaactgtaagaaa

SEQ ID NO: 76 - DNA, 17 bp, Artificial sequence
  Synthetic construct
gttgaaactgtgagaaa

SEQ ID NO: 77 - DNA, 16 bp, Artificial sequence
  Synthetic construct
gttaagactgcaagga

SEQ ID NO: 78 - DNA, 17 bp, Artificial sequence
  Synthetic construct
gttaaaactgtaagcgg

SEQ ID NO: 79 - DNA, 16 bp, Artificial sequence
  Synthetic construct
attgaaactgtaaaga

SEQ ID NO: 80 - DNA, 18 bp, Artificial sequence
  Synthetic construct
gtctgaaactgtaaacgg

SEQ ID NO: 81 - DNA, 17 bp, Artificial sequence
  Synthetic construct
gttgaagctgtaagcaa

SEQ ID NO: 82 - DNA, 16 bp, Artificial sequence
  Synthetic construct
gttgaatctgtacgga

SEQ ID NO: 83 - DNA, 18 bp, Artificial sequence
  Synthetic construct
gctgagattgtaaagtga

SEQ ID NO: 84 - DNA, 17 bp, Artificial sequence
  Synthetic construct
gttgggactgtgagcca

SEQ ID NO: 85 - DNA, 17 bp, Artificial sequence
  Synthetic construct
gttgatactgtgagcgg

SEQ ID NO: 86 - DNA, 17 bp, Artificial sequence
  Synthetic construct
gttgaaactgttagggg

SEQ ID NO: 87 - DNA, 16 bp, Artificial sequence
  Synthetic construct
gtagacgatgaagcga

SEQ ID NO: 88 - DNA, 17 bp, Artificial sequence
  Synthetic construct
attgaggccgtaagcaa

SEQ ID NO: 89 - DNA, 17 bp, Artificial sequence
  Synthetic construct
gtttaaaaccactttaa

SEQ ID NO: 90 - DNA, 17 bp, Artificial sequence
  Synthetic construct
gttaaataataagaaag

SEQ ID NO: 91 - DNA, 17 bp, Artificial sequence
  Synthetic construct
gttaaataataagaaaa

SEQ ID NO: 92 - DNA, 17 bp, Artificial sequence
  Synthetic construct
ataaaataataagaaag

SEQ ID NO: 93 - DNA, 16 bp, Artificial sequence
  Synthetic construct
gtctaacgaccttcta

SEQ ID NO: 94 - DNA, 17 bp, Artificial sequence
  Synthetic construct
ggctacataaagcctat

SEQ ID NO: 95 - DNA, 17 bp, Artificial sequence
  Synthetic construct
ggctacataaagcctgt

SEQ ID NO: 96 - DNA, 17 bp, Artificial sequence
  Synthetic construct
tgctacataaagcctgt

SEQ ID NO: 97 - DNA, 17 bp, Artificial sequence
  Synthetic construct
ggctacttaaagcctat

SEQ ID NO: 98 - DNA, 17 bp, Artificial sequence
  Synthetic construct
gcttagaacctttaaat

SEQ ID NO: 99 - DNA, 17 bp, Artificial sequence
  Synthetic construct
gcttaatcaacccttag

SEQ ID NO: 100 - DNA, 17 bp, Artificial sequence
  Synthetic construct
gtttaatcaacccttag

SEQ ID NO: 101 - DNA, 17 bp, Artificial sequence
  Synthetic construct
gtttaataatcctttag

SEQ ID NO: 102 - DNA, 17 bp, Artificial sequence
  Synthetic construct
gtctaaaggccttataa

SEQ ID NO: 103 - DNA, 18 bp, Artificial sequence
  Synthetic construct
gatttgaaagcatctttt

SEQ ID NO: 104 - DNA, 18 bp, Artificial sequence
  Synthetic construct
tatttgaaagcatctttt

SEQ ID NO: 105 - DNA, 18 bp, Artificial sequence
  Synthetic construct
aatttgaaagcatctttt

SEQ ID NO: 106 - DNA, 18 bp, Artificial sequence
  Synthetic construct
agtttgaaagcatatttt

SEQ ID NO: 107 - DNA, 18 bp, Artificial sequence
  Synthetic construct
gatttgaaagcatatttt

SEQ ID NO: 108 - DNA, 18 bp, Artificial sequence
  Synthetic construct
tatttggaagcacatttt

SEQ ID NO: 109 - DNA, 18 bp, Artificial sequence
  Synthetic construct
catttggaagcatatttt

SEQ ID NO: 110 - DNA, 18 bp, Artificial sequence
  Synthetic construct
aatttggaagcacatttt

SEQ ID NO: 111 - DNA, 18 bp, Artificial sequence
  Synthetic construct
tgtttggaagcatatttt

SEQ ID NO: 112 - DNA, 19 bp, Artificial sequence
  Synthetic construct
aatttctactcttgtagat

SEQ ID NO: 113 - DNA, 2801 bp, Artificial sequence
  Synthetic construct
gtgagccccacgttctgcttcactctccccatctcccccccctccccacccccaattttgtatttatttattttttaattattttgtgcagcgatgggggcggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgttgccttcgccccgtgccccgctccgcgccgcctcgcgccgcccgccccggctctgactgaccgcgttactcccacaggtgagcgggcgggacggcccttctcctccgggctgtaattagcgcttggtttaatgacggctcgtttcttttctgtggctgcgtgaaagccttaaagggctccgggagggccctttgtgcgggggggagcggctcggggggtgcgtgcgtgtgtgtgtgcgtggggagcgccgcgtgcggcccgcgctgcccggcggctgtgagcgctgcgggcgcggcgcggggctttgtgcgctccgcgtgtgcgcgaggggagcgcggccgggggcggtgccccgcggtgcgggggggctgcgaggggaacaaaggctgcgtgcggggtgtgtgcgtgggggggtgagcagggggtgtgggcgcggcggtcgggctgtaacccccccctgcacccccctccccgagttgctgagcacggcccggcttcgggtgcggggctccgtgcggggcgtggcgcggggctcgccgtgccgggcggggggtggcggcaggtgggggtgccgggcggggcggggccgcctcgggccggggagggctcgggggaggggcgcggcggccccggagcgccggcggctgtcgaggcgcggcgagccgcagccattgccttttatggtaatcgtgcgagagggcgcagggacttcctttgtcccaaatctggcggagccgaaatctgggaggcgccgccgcaccccctctagcgggcgcgggcgaagcggtgcggcgccggcaggaaggaaatgggcggggagggccttcgtgcgtcgccgcgccgccgtccccttctccatctccagcctcggggctgccgcagggggacggctgccttcgggggggacggggcagggcggggttcggcttctggcgtgtgaccggcggctctagagcctctgctaaccatgttcatgccttcttctttttcctacagctcctgggcaacgtgctggttgttgtgctgtctcatcattttggcaaagaattgaattcgtcgccaccatggagctgattaaggagaacatgcacatgaagctgtacatggagggcaccgtggacaaccatcacttcaagtgcacatccgagggcgaaggcaagccctacgagggcacccagaccatgagaatcaaggtggtcgagggcggccctctccccttcgccttcgacatcctggctactagcttcctctacggcagcaagaccttcatcaaccacacccagggcatccccgacttcttcaagcagtccttccctgagggcttcacatgggagagagtcaccacatacgaagacgggggcgtgctgaccgctacccaggacaccagcctccaggacggctgcctcatctacaacgtcaagatcagaggggtgaacttcacatccaacggccctgtgatgcagaagaaaacactcggctgggaggccttcaccgagacgctgtaccccgctgacggcggcctggaaggcagaaacgacatggccctgaagctcgtgggcgggagccatctgatcgcaaacatcaagaccacatatagatccaagaaacccgctaagaacctcaagatgcctggcgtctactatgtggactacagactggaaagaatcaaggaggccaacaacgagacctacgtcgagcagcacgaggtggcagtggccagatactgcgacctccctagcaaactggggcacaagcttaattaagaattcggatcacgcgtgattcgtcagtagggttgtaaaggtttttcttttcctgagaaaacaaccttttgttttctcaggttttgctttttggcctttccctagctttaaaaaaaaaaaagcaaaaggtacctgatttaattgcaaatctttgaaataatgcagacttaaatttataaattcatggaataaggtgattttattgtgaaaaaatactcgtattttgttggaaaaacatctttttgttgtataatatgatgatatacgggatcctttctttctttaaataatttctactaagtgtagataaaagtgccactccttagggaaataatttctactaagtgtagatagatgagatggtgacagataaaataatttctactaagtgtagatctccagagcccgactcgccgaaataatttctactaagtgtagattcggcttgcggggagacttcaaataatttctactaagtgtagatcacccttcggcccgccacccaaataatttctactaagtgtagatagaaagggaatcccagggccaaataatttctactaagtgtagatctccctatcagtgatagagaaaataatttctactaagtgtagatgttaacttgtttattgcagcttataatggttacaaataaagcaatagcatcacaaatttcacaaataaagcatttttttcactgcattctagttgtggtttgtccaaactcatcaatgtatcttatcatgtctggatc

SEQ ID NO: 114 - DNA, 4 bp, Artificial sequence
  Synthetic construct
tttt

SEQ ID NO: 115 - DNA, 4 bp, Artificial sequence
  Synthetic construct
ttta

SEQ ID NO: 116 - DNA, 4 bp, Artificial sequence
  Synthetic construct
attt

SEQ ID NO: 117 - DNA, 2653 bp, Artificial sequence
  Synthetic construct
gtgagccccacgttctgcttcactctccccatctcccccccctccccacccccaattttgtatttatttattttttaattattttgtgcagcgatgggggcggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgttgccttcgccccgtgccccgctccgcgccgcctcgcgccgcccgccccggctctgactgaccgcgttactcccacaggtgagcgggcgggacggcccttctcctccgggctgtaattagcgcttggtttaatgacggctcgtttcttttctgtggctgcgtgaaagccttaaagggctccgggagggccctttgtgcgggggggagcggctcggggggtgcgtgcgtgtgtgtgtgcgtggggagcgccgcgtgcggcccgcgctgcccggcggctgtgagcgctgcgggcgcggcgcggggctttgtgcgctccgcgtgtgcgcgaggggagcgcggccgggggcggtgccccgcggtgcgggggggctgcgaggggaacaaaggctgcgtgcggggtgtgtgcgtgggggggtgagcagggggtgtgggcgcggcggtcgggctgtaacccccccctgcacccccctccccgagttgctgagcacggcccggcttcgggtgcggggctccgtgcggggcgtggcgcggggctcgccgtgccgggcggggggtggcggcaggtgggggtgccgggcggggcggggccgcctcgggccggggagggctcgggggaggggcgcggcggccccggagcgccggcggctgtcgaggcgcggcgagccgcagccattgccttttatggtaatcgtgcgagagggcgcagggacttcctttgtcccaaatctggcggagccgaaatctgggaggcgccgccgcaccccctctagcgggcgcgggcgaagcggtgcggcgccggcaggaaggaaatgggcggggagggccttcgtgcgtcgccgcgccgccgtccccttctccatctccagcctcggggctgccgcagggggacggctgccttcgggggggacggggcagggcggggttcggcttctggcgtgtgaccggcggctctagagcctctgctaaccatgttcatgccttcttctttttcctacagctcctgggcaacgtgctggttgttgtgctgtctcatcattttggcaaagaattgaattcgtcgccaccatggagctgattaaggagaacatgcacatgaagctgtacatggagggcaccgtggacaaccatcacttcaagtgcacatccgagggcgaaggcaagccctacgagggcacccagaccatgagaatcaaggtggtcgagggcggccctctccccttcgccttcgacatcctggctactagcttcctctacggcagcaagaccttcatcaaccacacccagggcatccccgacttcttcaagcagtccttccctgagggcttcacatgggagagagtcaccacatacgaagacgggggcgtgctgaccgctacccaggacaccagcctccaggacggctgcctcatctacaacgtcaagatcagaggggtgaacttcacatccaacggccctgtgatgcagaagaaaacactcggctgggaggccttcaccgagacgctgtaccccgctgacggcggcctggaaggcagaaacgacatggccctgaagctcgtgggcgggagccatctgatcgcaaacatcaagaccacatatagatccaagaaacccgctaagaacctcaagatgcctggcgtctactatgtggactacagactggaaagaatcaaggaggccaacaacgagacctacgtcgagcagcacgaggtggcagtggccagatactgcgacctccctagcaaactggggcacaagcttaattaagaattcggatcacgcgtgattcgtcagtagggttgtaaaggtttttcttttcctgagaaaacaaccttttgttttctcaggttttgctttttggcctttccctagctttaaaaaaaaaaaagcaaaaggtacctgatttaattgcaaatctttgaaataatgcagacttaaatttataaattcatggaataaggtgattttattgtgaaaaaatactcgtattttgttggaaaaacatctttttgttgtataatatgatgatatacgggatcctttctttcaagtaaacccctaccaactggtcggggtttgaaacggtgctcaggtagtggttgtcgggaaataatttctactaagtgtagataaaagtgccactccttagggcaagtaaacccctaccaactggtcggggtttgaaacatgtggtcggggtagcggctgaagaaataatttctactaagtgtagatgttaacttgtttattgcagcttataatggttacaaataaagcaatagcatcacaaatttcacaaataaagcatttttttcactgcattctagttgtggtttgtccaaactcatcaatgtatcttatcatgtctggatc

SEQ ID NO: 118 - DNA, 336 bp, Artificial sequence
  Synthetic construct
caagtaaacccctaccaactggtcggggtttgaaacatgtggtcggggtagcggctgaagcaagtaaacccctaccaactggtcggggtttgaaacctgtactggtggatgtcctcaaaacaagtaaacccctaccaactggtcggggtttgaaacctggtgaggattccagtcgctgtcaaataatttctactaagtgtagataaaagtgccactccttagggaaataatttctactaagtgtagatagatgagatggtgacagataaaataatttctactaagtgtagatcaggagggtgactcaggctaaaataatttctactaagtgtagat

SEQ ID NO: 119 - DNA, 482 bp, Artificial sequence
  Synthetic construct
caagtaaacccctaccaactggtcggggtttgaaacatgtggtcggggtagcggctgaagcaagtaaacccctaccaactggtcggggtttgaaacctgtactggtggatgtcctcaaaacaagtaaacccctaccaactggtcggggtttgaaacctggtgaggattccagtcgctgtccaagtaaacccctaccaactggtcggggtttgaaacgattcgtcagtagggttgtaaaggtttttcttttcctgagaaaacaaccttttgttttctcaggttttgctttttggcctttccctagctttaaaaaaaaaaaagcaaaaaaataatttctactaagtgtagataaaagtgccactccttagggaaataatttctactaagtgtagatagatgagatggtgacagataaaataatttctactaagtgtagatcaggagggtgactcaggctaaaataatttctactaagtgtagat

SEQ ID NO: 120 - DNA, 348 bp, Artificial sequence
  Synthetic construct
aaataatttctactaagtgtagataaaagtgccactccttagggaaataatttctactaagtgtagatagatgagatggtgacagataaaataatttctactaagtgtagatcaggagggtgactcaggctacaagtaaacccctaccaactggtcggggtttgaaacatgtggtcggggtagcggctgaagcaagtaaacccctaccaactggtcggggtttgaaacctgtactggtggatgtcctcaaaacaagtaaacccctaccaactggtcggggtttgaaacctggtgaggattccagtcgctgtccaagtaaacccctaccaactggtcggggtttgaaac

SEQ ID NO: 121 - DNA, 482 bp, Artificial sequence
  Synthetic construct
aaataatttctactaagtgtagataaaagtgccactccttagggaaataatttctactaagtgtagatagatgagatggtgacagataaaataatttctactaagtgtagatcaggagggtgactcaggctaaaataatttctactaagtgtagatgattcgtcagtagggttgtaaaggtttttcttttcctgagaaaacaaccttttgttttctcaggttttgctttttggcctttccctagctttaaaaaaaaaaaagcaaaacaagtaaacccctaccaactggtcggggtttgaaacatgtggtcggggtagcggctgaagcaagtaaacccctaccaactggtcggggtttgaaacctgtactggtggatgtcctcaaaacaagtaaacccctaccaactggtcggggtttgaaacctggtgaggattccagtcgctgtccaagtaaacccctaccaactggtcggggtttgaaac

SEQ ID NO: 122 - DNA, 348 bp, Artificial sequence
  Synthetic construct
caagtaaacccctaccaactggtcggggtttgaaacatgtggtcggggtagcggctgaagaaataatttctactaagtgtagataaaagtgccactccttagggcaagtaaacccctaccaactggtcggggtttgaaacctgtactggtggatgtcctcaaaaaaataatttctactaagtgtagatagatgagatggtgacagatacaagtaaacccctaccaactggtcggggtttgaaacctggtgaggattccagtcgctgtcaaataatttctactaagtgtagatcaggagggtgactcaggctacaagtaaacccctaccaactggtcggggtttgaaac